SEQUENCE LISTING (17 sequences)

SEQ ID NO: 1 - protein, 745 aa, synthetic construct
  source 1..745: note = Rh74Myo VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQENRRGDFNNTAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 10 aa, synthetic construct
  source 1..10: note = 4E insert
ENRRGDFNNT

SEQ ID NO: 3 - DNA, 2238 bp, synthetic construct
  source 1..2238: mol_type = other DNA; note = Rh74Myo
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaagaaaacaggagaggagacttcaacaacactgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 4 - protein, 738 aa, Dependoparvovirus sp.
  source 1..738: note = Rh74 wt VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 5 - protein, 17 aa, synthetic construct
  source 1..17: note = Rh74 HVR4-4E
LSRTENRRGDFNNTQLL

SEQ ID NO: 6 - protein, 16 aa, synthetic construct
  source 1..16: note = Rh74 HVR4-AAAV9
LSRTINGSGQNQQTLL

SEQ ID NO: 7 - protein, 1192 aa, synthetic construct
  source 1..1192: note = Human uDys
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 8 - DNA, 3579 bp, synthetic construct
  source 1..3579: mol_type = other DNA; note = Human uDys
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtga

SEQ ID NO: 9 - protein, 1192 aa, synthetic construct
  source 1..1192: note = NHP uDys
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVATTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKEQFHTHEGYMMDLTAHQGRVGNILQLGSQLIGTGKLSEDEETEVQEQMNLLNSRWECLRVASMEKQSNLHRVLMDLQNQKLKELNDWLTKTEERTRKMEKEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDVVNRIHTTGFKDQNEMLSSLQKLAVLKADLEKKKQSMDKLYSLKQDLLSTLKNKSVTQKMEAWLENFARCWDNLVQKLEKSTAQISQAVTTTQPSLTQTTVMETVTMVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVSSSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 10 - DNA, 3579 bp, synthetic construct
  source 1..3579: mol_type = other DNA; note = NHP uDys
atgctctggtgggaagaagttgaagactgctacgagcgcgaagatgtacaaaagaagaccttcaccaaatgggtcaatgcacagttttccaaatttggtaaacagcacatcgagaaccttttctctgatctgcaggatggcagacggctgctggacctgctagaggggctgactggtcagaaactccccaaggagaaaggttctacacgcgtccatgcgcttaacaatgtgaataaggctctccgagtacttcagaacaataatgtagatttggtgaatataggatctactgatattgttgacggaaaccacaaactgacattaggtctgatctggaatattatcctgcactggcaggtgaaaaatgtgatgaagaacatcatggccggcctccaacagaccaattcagaaaagatattacttagctgggtgaggcagagcactaggaactacccccaggttaatgtaattaatttcacaacgagctggagcgatgggctcgcactgaatgcgttgatccactcccacagacctgatttgtttgactggaactcggttgtctgtcagcaatccgccactcagcggctggagcatgccttcaacattgcaagatatcagctcgggattgaaaaattactcgatccagaggacgtggccaccacctacccagacaagaagagcatcctcatgtacatcactagcctcttccaagttttgcctcaacaggtcagcattgaggccattcaggaggtggagatgctgcccaggcctccaaaggtcactaaagaggagcattttcaactccaccaccagatgcattatagtcagcagataaccgtgtcgttagctcagggttatgagaggaccagtagccctaagccccgcttcaagtcctacgcttacacccaggctgcctatgtcacaactagcgacccaactaggtcaccgttcccgtcacaacaccttgaagcccccgaggataaatcttttggaagttccctgatggaatcagaagtgaacttggacaggtaccagactgctttagaagaggtcctgtcctggctgctgagtgccgaggacaccctgcaagcacaaggtgaaatatcgaatgacgtcgaggtggttaaagaacagttccacactcatgagggctacatgatggatctcacagcacaccagggccgggtgggaaacattctgcagctgggaagtcagctcataggaacaggcaagctcagtgaagacgaggaaacagaagtacaggagcagatgaacctgcttaacagcaggtgggagtgtctgcgggttgcatcaatggagaaacagagtaacctgcatcgagtcttgatggatttacaaaaccagaaactgaaagaactgaacgattggctgacgaagacggaggagcgtactcgcaagatggagaaggagccgcttggaccagacctcgaagacctgaagcgacaggtgcagcagcataaagtgctgcaggaagatcttgagcaggaacaagtgcgggtaaattccctgacacacatggtcgtagtggtggatgagagcagtggagaccacgccacagctgctctggaggaacagttaaaagtgctgggcgaccgctgggccaatatttgtagatggactgaggaccgatgggttctattgcaggacatattgctaaagtggcagcgtctaaccgaggaacagtgcctgttttctgcctggctgtctgaaaaggaagatgtggtgaacagaattcataccactggatttaaagatcagaatgaaatgctgtcctcgctacagaagctggctgtgctgaaagctgacctagagaagaaaaaacagtcaatggacaaactctactccctaaaacaagacttgctgagtacactgaagaacaagtctgttacccagaaaatggaggcctggttagaaaattttgcccgctgctgggacaacctagtccagaagctggaaaagtccacagcccagatctcccaggccgtgaccactactcagccatcacttactcagacaaccgtcatggaaactgtcaccatggtaacaacgcgggagcagatactggtcaagcatgctcaagaagaactgcctcccccgcctccccaaaagaaaaggactttggagcggttgcaagaactccaagaggcaaccgatgagcttgacctaaaactccgacaagctgaagtgattaaagggtcttggcagcccgtgggggacctcctgatagattctttgcaagatcatttagaaaaagtcaaagccctgagaggtgagattgctccattgaaggaaaacgtgtctcatgttaatgacttagcgagacagctcacgacccttgggatccagttgtccccctataatctttcgacattggaggacctcaacacgcgttggaagcttctgcaagttgcagtagaagatagagtccgtcagctgcatgaggcgcacagggactttggcccagcgtctcagcactttttgagcacatccgttcaggggccctgggagcgcgccatctcacccaacaaagttccatactatattaaccatgagactcaaaccacctgctgggatcaccctaaaatgaccgagctctatcaatctctggcagatttgaataatgtgcggttctcagcatatcgaacagccatgaaattacgccgcctccaaaaggcgctttgcttggatctgctctctctcagcgcagcctgtgatgccctcgaccagcataatttgaagcagaacgatcagcctatggatatcttacaaatcatcaactgcctaacgaccatctatgatagacttgagcaggaacacaacaacctggtgaatgtgcccctctgtgtggacatgtgtctgaactggctcctcaatgtctatgacacagggaggaccggccgcatccgagttctgagttttaagacaggaattatctccctttgtaaagcccacctggaggataagtacaggtacctgttcaagcaagtttcatcttccaccggcttttgtgaccagagaagactcggtttactgcttcatgatagtatacaaatccctagacagttgggagaggtggcctcttttgggggcagcaacattgagccctctgtcagaagctgcttccagtttgccaataataaacctgaaattgaagcagctttgttcctggactggatgagcttggagccacagagcatggtgtggctgcctgtcctgcaccgagtagccgctgcagagacagcaaagcatcaggcgaaatgcaatatctgcaaggagtgtccgatcattgggttccggtaccggtcactgaagcacttcaattatgatatctgccagagttgtttcttcagcgggcgggtagccaagggccacaagatgcactatcctatggtcgagtactgcaccccaactaccagcggcgaggacgtgagagattttgctaaagtgctaaagaacaaatttagaacaaaacgctacttcgctaagcatccaaggatgggctatcttcctgtgcaaacagtgttggaaggagacaacatggaaacagacaccatgtga

SEQ ID NO: 11 - protein, 738 aa, synthetic construct
  source 1..738: note = Rh74-HVR4.4E
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTENRRGDFNNTQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 12 - DNA, 792 bp, synthetic construct
  source 1..792: mol_type = other DNA; note = MHCK7 muscle-specific promoter/enhancer
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 13 - DNA, 150 bp, synthetic construct
  source 1..150: mol_type = other DNA; note = SV40 intron
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgc

SEQ ID NO: 14 - DNA, 145 bp, synthetic construct
  source 1..145: mol_type = other DNA; note = 5' AAV2 ITR
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 15 - DNA, 145 bp, synthetic construct
  source 1..145: mol_type = other DNA; note = 3' AAV2 ITR
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 16 - DNA, 53 bp, synthetic construct
  source 1..53: mol_type = other DNA; note = polyadenylation (PolyA) signal
ggccgcaataaaagatctttattttcattagatctgtgtgttggttttttgtg

SEQ ID NO: 17 - DNA, 4977 bp, synthetic construct
  source 1..4977: mol_type = other DNA; note = Microdystrophin Expression Cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

(19) United States
(12) Patent Application Publication
(10) Pub. No.: US 2025/0369016 A1
(43) Pub. Date: Dec. 4, 2025

(54) MUSCLE TROPIC RAAV

(71) Applicant: Sarepta Therapeutics, Inc., Cambridge, MA (US)

(72) Inventors: John SNEDEKER, Cambridge, MA (US); Louise RODINO-KLAPAC, Cambridge, MA (US); Rachael POTTER, Cambridge, MA (US); Arani CHANDA, Cambridge, MA (US); Shon CRAMPTON, Cambridge, MA (US); Pooja SANE, Cambridge, MA (US)

(73) Assignee: Sarepta Therapeutics, Inc., Cambridge, MA (US)

(21) Appl. No.: 19/223,409

(22) Filed: May 30, 2025

Related U.S. Application Data
(60) Provisional application No. 63/654,224, filed on May 31, 2024.
(60) Provisional application No. 63/660,638, filed on Jun. 17, 2024.

(51) Int. Cl. C12N 15/86; A61K 38/17; A61K 47/02; A61K 47/10; A61K 47/18; A61K 47/26; A61K 48/00; A61P 21/00; C07K 14/47

(52) U.S. Cl. CPC C12N 15/86; A61K 38/1719; A61K 47/02; A61K 47/10; A61K 47/18; A61K 47/26; A61K 48/0058; A61P 21/00; C07K 14/4716; C12N 2750/14122; C12N 2750/14143; C12N 2830/008; C12N 2830/42; C12N 2830/50

(57) ABSTRACT

Recombinant myotropic AAV rh.74 capsids are described herein. In addition, plasmids, viral vectors, dual vector systems, cells, and compositions comprising or encoding such recombinant capsids are further described. Such recombinant polynucleotides, plasmids, viral vectors, dual vector systems, cells, and compositions may be used to treat muscle diseases or disorders, including muscular dystrophies and cardiomyopathies.

### CROSS-REFERENCE TO RELATED APPLICATIONS

This application claims the benefit of U.S. Provisional Appl. No. 63/654,224, filed May 31, 2024, and U.S. Provisional Appl. No. 63/660,638, filed Jun. 17, 2024, the contents of which are hereby incorporated by reference in their entireties.

### REFERENCE TO SEQUENCE LISTING SUBMITTED ELECTRONICALLY

The content of the electronically submitted sequence listing in text format (Name: 4140_1430002_SequenceListing_ST26.xml; Size: 39,006 bytes; and Date of Creation: May 27, 2025) filed with the application is incorporated by reference in its entirety.

### TECHNICAL FIELD

This disclosure provides viral vectors, polynucleotides encoding the viral vectors, cells transduced with the viral vectors, and compositions comprising such viral vectors, methods of using such viral vectors, and compositions to treat subjects with genetic muscle disorders, and methods of production of the viral vectors.

### BACKGROUND

Therapeutics capable of providing functional gene replacements or means of editing defective genes promise relief from disorders caused by single gene mutations, many of which have no current hope of treatment other than palliative care. Targeted delivery of genetic medicines is required to optimize efficacy while minimizing potential adverse events associated with off-target gene expression.

Recombinant adeno-associated viruses (rAAV) are frequently proposed as vectors for gene therapies. (Wang, D. et al., Nature Reviews 18:358-378 (2019)). However, rAAV also pose several problems that need to be solved for each therapeutic (Au 2022). Such problems include:
The limited size (˜4.7 kb single-stranded DNA) of the rAAV genetic material, which must include inverted terminal repeats (ITR) necessary for packaging and replication of the genetic cassette, promoters, at least one gene of interest, and transcription terminators. Defects in large host genes and in multiple genes are thus difficult to treat with rAAV vectors.
Pre-existing antibodies to the rAAV capsid may prevent the therapeutic from transducing an effective number of cells in the afflicted tissue.
The rAAV may infect a variety of different cell types, not just cells in the afflicted tissue, leading to off-target effects. Liver pathology is often observed. Tissue-specific promoters may prevent such effects, but identification of the optimal promoters may be challenging.
Large numbers of rAAV particles are needed to deliver effective treatment to human, typically in the range of 10¹⁴ to 10¹⁶ viral genomes (vg) for systemic (e.g. intravenous) administration, yet rAAV manufacture is limited by current technologies.

Treatment of diseases and disorders of the muscle, including striated/skeletal muscle and cardiac (smooth) muscle, requires vectors which preferentially target muscle cells. Such targeting would both reduce off-target effects and perhaps decrease the number of rAAV particles needed. Peptide motifs which improve myotropic properties of recombinant adeno-associated virus vectors have previously been described, such as in WO2021/077000 and WO2022/020616. However, these viral vectors are on the AAV9 background, and some of these AAV variants present production difficulties and safety concerns. Thus, there is a need for improved myotropic AAV vectors for the treatment of muscle diseases and disorders.

### SUMMARY

Disclosed herein is a myotropic AAV rh74 variants and of using the same.

In one embodiment, an amino acid sequence of ENRRGDFNNT (SEQ ID NO: 2) is inserted in a wild type AAVrh74 capsid protein. The amino acid sequence may be inserted in the hypervariable regions IV (four) and/or VIII (eight) of the rh74 capsid. Based on the insertion site, the amino acid sequence may be in a VP1, a VP2, and/or a VP3 capsid protein.

In one embodiment, an amino acid sequence of ENRRGDFNNT (SEQ ID NO: 2) is inserted in a wild type AAVrh74 capsid protein (SEQ ID NO: 4) between positions 587 and 591. SEQ ID NO: 2 may be inserted in SEQ ID NO: 4 such that amino acids 588, 589, and 590 of the capsid protein are replaced by SEQ ID NO: 2. A modified rh74 capsid may have an amino sequence at least 95%, at least 96%, at least 97%, at least 98%, at least 99%, at least 99.5%, or 100% identical to SEQ ID NO: 1. A nucleotide encoding the modified rh74 capsid may have an amino sequence at least 95%, at least 96%, at least 97%, at least 98%, at least 99%, at least 99.5%, or 100% identical to SEQ ID NO: 3.

In one embodiment, an amino acid sequence of ENRRGDFNNT (SEQ ID NO: 2) is inserted in an AAVrh74 capsid protein (SEQ ID NO: 4) between positions 451 and 462. SEQ ID NO: 2 may be inserted in SEQ ID NO: 4 such that amino acids 452-461 of the capsid protein are replaced by SEQ ID NO: 2. A modified rh74 capsid may have an amino sequence at least 95%, at least 96%, at least 97%, at least 98%, at least 99%, at least 99.5% or 100% identical to SEQ ID NO: 11.

In one embodiment, the modified AAVrh74 capsid protein may have at least one additional modification compared to the wild type AAVrh74 capsid protein. The additional mutation or mutations may be a substitution, a deletion, and/or an insertion.

In one embodiment, a modified AAV particle may comprise any of the modified AAVrh74 capsid proteins described herein and a transfer cassette polynucleotide. The transfer cassette may be encapsulated by the modified rh74 capsid protein. The modified AAV particle may comprise at least one modified and at least one wild type AAVrh74 capsid protein. The transfer cassette may comprise one or more of an inverted terminal repeat (ITR), a promoter, an intron, a transgene and a polyA signal sequence. The transgene may be a microdystrophin, a dysferlin, or an anoctamin-5 transgene.

In one embodiment, a modified AAV particle may comprise any of the modified AAVrh74 capsid proteins described herein and a transfer cassette polynucleotide encapsulated by the modified rh74 capsid protein. The transfer cassette may comprise a microdystrophin transgene. The microdystrophin transgene may be a human or nonhuman primate transgene. The transgene may have a nucleotide sequence at least 95%, at least 96%, at least 97%, at least 98%, at least 99%, at least 99.5% or 100% identical to SEQ ID NO: 8 or SEQ ID NO: 10. The transgene may encode a protein having a sequence at least 95%, at least 96%, at least 97%, at least 98%, at least 99%, at least 99.5%, or 100% identical to SEQ ID NO: 7 or SEQ ID NO: 9.

In one embodiment, a modified AAV particle may comprise any of the modified AAVrh74 capsid proteins described herein and a transfer cassette polynucleotide encapsulated by the modified rh74 capsid protein. The transfer cassette may comprise a promoter. The transfer cassette may comprise a muscle-specific promoter. The muscle-specific promoter may be from a human skeletal actin gene element, a cardiac actin gene element, a desmin promoter, a skeletal alpha-actin (ASKA) promoter, a troponin I (TNNI2) promoter, a myocyte-specific enhancer binding factor, mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor.

In one embodiment, a plasmid may contain a nucleotide sequence encoding any of the modified AAVrh74 capsid proteins described herein. In another embodiment, a plasmid may contain a nucleotide sequence encoding any of the transfer cassettes described herein.

In one embodiment, a vector system may include a plasmid containing a nucleotide sequence encoding any of the modified AAVrh74 capsid proteins described herein and/or a plasmid containing a nucleotide sequence encoding any of the transfer cassettes described herein. The nucleotides encoding the modified rh74 capsid protein and the nucleotides comprising the transfer cassette sequence may be on the same plasmid or on different plasmids. The vector system may include a cell transfected with any of the nucleotides or plasmids described herein. The cell may also be transfected with an adenovirus-derived helper plasmid. The cell may also stably express adenovirus-derived helper factors and/or modified rh74 capsid protein.

In one embodiment, a mammalian cell may be transduced by any of the modified AAV particles described herein.

In one embodiment, a pharmaceutical composition may contain any of the modified AAV capsid proteins described herein, any of the modified AAV particles described herein, or any combination thereof, and a pharmaceutically acceptable carrier. In one embodiment, a pharmaceutical composition may contain about 20 mM Tris pH 8, about 1 mM MgCl₂, about 160-220 mM NaCl, a surfactant and optionally 1-3% sucrose. The sodium chloride may be present at 160 mM, 170 mM, 180 mM, 190 mM, 200 mM, 210 mM or 220 mM. The surfactant may be a poloxamer at 0.01% to 0.2% or polysorbate 80 at 0.0001% to 0.005%. The poloxamer may be present at 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, or 0.2%. The poloxamer may be P188. The polysorbate 80 (or Tween 80) may be present at 0.0001%, 0.0002%, 0.0004%, 0.0006%, 0.0008%, 0.001%, 0.0013%, 0.0016%, 0.002%, 0.0023%, 0.0026%, 0.003%, 0.0033%, 0.0036%, 0.004%, 0.0043%, 0.0046%, or 0.005%. The sucrose may be present at 1%, 1.2%, 1.4%, 1.6%, 1.8%, 2%, 2.2%, 2.4%, 2.6%, 2.8%, or 3%. In another embodiment, a pharmaceutical composition may contain about 20 mM Tris pH 8, about 1 mM MgCl₂, about 180-200 mM NaCl, and about 0.01%-0.1% poloxamer and optionally about 2% sucrose. In another embodiment, a pharmaceutical composition may contain 20 mM Tris pH 8, 1 mM MgCl₂, 180 mM NaCl, 0.1% poloxamer and 2% sucrose. In another embodiment, a pharmaceutical composition may contain 20 mM Tris pH 8, 1 mM MgCl₂, 200 mM NaCl, 0.1% poloxamer and 2% sucrose. In another embodiment, a pharmaceutical composition may contain 20 mM Tris pH 8, 1 mM MgCl₂, 200 mM NaCl, 0.01% poloxamer and 2% sucrose. In another embodiment, a pharmaceutical composition may contain 20 mM Tris pH 8, 1 mM MgCl₂, 200 mM NaCl, and 0.01% poloxamer. Any pharmaceutical composition described herein may contain any of the modified AAV capsid proteins described herein and/or any of the modified AAV particles described herein.

In one embodiment, a method of treating a muscular disease may include administering to a subject in need thereof a therapeutically effective amount of any of the modified AAV capsid proteins described herein, any of the modified AAV particles described herein, any of the pharmaceutical compositions described herein, or any combination thereof. In one embodiment, a method of treating a muscular disease may include treating a Duchenne muscular dystrophy, Becker's muscular dystrophy, Myotonic muscular dystrophy, Facioscapulohumeral muscular dystrophy, a limb-girdle muscle dystrophy, or a cardiomyopathy. The treatment may be provided to a human or a nonhuman primate.

In one embodiment, the dose of modified AAV particles used in a method of treating a muscular disease may be between about 1×10¹³ and about 2×10¹⁴ vector genomes per kilogram body weight (vg/kg). In one embodiment, the dose of modified AAV particles used in a method of treating a muscular disease may be between about 1×10¹³ and about 5×10¹³ vg/kg. In one embodiment, the dose of modified AAV particles used in a method of treating a muscular disease may be between about 1×10¹³, about 1.5×10¹³, about 2×10¹³, about 2.25×10¹³, about 2.5×10¹³, about 2.75×10¹³, about 3×10¹³, about 3.5×10¹³, about 4×10¹³, about 4.5×10¹³, and about 5×10¹³ vg/kg. In one embodiment, the dose of modified AAV particles used in a method of treating a muscular disease may be between about 2×10¹³ and about 3×10¹³ vg/kg.

In one embodiment, any of the modified AAV capsid proteins described herein, any of the modified AAV particles described herein, any of the pharmaceutical compositions described herein, or any combination thereof may be used in the manufacture of a medicament to treat a muscle disease or disorder. Another embodiment includes use of any of the modified AAV capsid proteins described herein, any of the modified AAV particles described herein, any of the pharmaceutical compositions described herein, or any combination thereof in the manufacture of a medicament to treat a Duchenne muscular dystrophy, Becker's muscular dystrophy, Myotonic muscular dystrophy, Facioscapulohumeral muscular dystrophy, a limb-girdle muscle dystrophy, or a cardiomyopathy.

In one embodiment, any of the modified AAV capsid proteins described herein, any of the modified AAV particles described herein, any of the pharmaceutical compositions described herein, or any combination thereof may be used in the therapy for a muscle disease or disorder. Another embodiment includes use of any of the modified AAV capsid proteins described herein, any of the modified AAV particles described herein, any of the pharmaceutical compositions described herein, or any combination thereof in therapy for a Duchenne muscular dystrophy, Becker's muscular dystrophy, Myotonic muscular dystrophy, Facioscapulohumeral muscular dystrophy, a limb-girdle muscle dystrophy, or a cardiomyopathy.

### ASPECTS

Aspect 1. A modified AAVrh74 capsid protein comprising the amino acid sequence ENRRGDFNNT (SEQ ID NO: 2) in a AAVrh74 capsid protein.

Aspect 2. The modified AAVrh74 capsid protein of aspect 1, wherein the polypeptide of amino acid sequence ENRRGDFNNT (SEQ ID NO: 2) is located in the HVR-VIII domain of the AAVrh74 capsid protein.

Aspect 3. The modified AAVrh74 capsid protein of aspect 1 or 2, wherein the AAVrh74 capsid protein is a VP1 capsid protein, a VP2 capsid protein, or a VP3 capsid protein.

Aspect 4. The modified AAVrh74 capsid protein of any one of aspects 1-3, wherein the polypeptide of amino acid sequence ENRRGDFNNT (SEQ ID NO: 2) is located between amino acid residues corresponding to amino acids 587 and 591 of a wild type VP1 AAVrh74 capsid protein (SEQ ID NO: 4); and wherein amino acid residues corresponding to amino acids 588, 589, and 590 of the wild type VP1 AAVrh74 capsid protein are absent.

Aspect 5. A modified AAVrh74 capsid protein comprising an AAVrh74 capsid protein comprising an amino acid sequence at least 95% identical to the amino acid sequence of SEQ ID NO: 1.

Aspect 6. The modified AAVrh74 capsid protein of aspect 1, wherein the polypeptide of amino acid sequence ENRRGDFNNT (SEQ ID NO: 2) is located in the HVR-IV domain of the AAVrh74 capsid protein.

Aspect 7. The modified AAVrh74 capsid protein of aspect 6, wherein the AAVrh74 capsid protein is a VP1 capsid protein, a VP2 capsid protein, or a VP3 capsid protein.

Aspect 8. The modified AAVrh74 capsid protein of aspect 7 or 8 wherein the polypeptide of amino acid sequence ENRRGDFNNT (SEQ ID NO: 2) is located between amino acid residues corresponding to amino acids 451 and 462 of SEQ ID NO:4, and wherein amino acid residues corresponding to amino acids 452-461 of the SEQ ID NO: 4 are absent.

Aspect 9. A modified AAVrh74 capsid protein comprising an AAVrh74 capsid protein comprising an amino acid sequence at least 95% identical to the amino acid sequence of SEQ ID NO: 11.

Aspect 10. The modified AAVrh74 capsid protein of aspect 1, comprising at least one additional modification compared to the AAVrh74 capsid protein.

Aspect 11. The modified AAVrh74 capsid protein of aspect 10, wherein the at least one additional modification is an amino acid substitution, deletion or insertion.

Aspect 12. The modified AAV capsid of any one of aspects 1-11, wherein the modified AAV capsid has increased tropism to muscle cells compared to a wild type AAVrh74 capsid when administered to mice.

Aspect 13. A modified AAV particle comprising (i) the modified AAVrh74 capsid protein of any one of aspects 1-12, and (ii) a transfer cassette.

Aspect 14. The modified AAV particle of aspect 13, wherein the AAV capsid encapsulates the transfer cassette.

Aspect 15. The modified AAV particle of aspect 13 or 14, wherein the transfer cassette comprises a recombinant polynucleotide comprising one or more nucleotide sequences selected from an inverted terminal repeat (ITR), a promoter, an intron, a transgene and a poly A signal sequence.

Aspect 16. The modified AAV particle of aspect 15, wherein the transgene comprises a microdystrophin, a dysferlin, an anoctamin-5 transgene, or any fragment thereof.

Aspect 17. The modified AAV particle of aspect 15, wherein the transgene comprises a human microdystrophin or a nonhuman primate microdystrophin.

Aspect 18. The modified AAV particle of aspect 17, wherein the human microdystrophin transgene encodes a protein having a sequence of SEQ ID NO: 7.

Aspect 19. The modified AAV particle of aspect 17, wherein the human microdystrophin transgene has a sequence of SEQ ID NO: 8.

Aspect 20. The modified AAV particle of aspect 17, wherein the nonhuman primate microdystrophin transgene encodes a protein having a sequence of SEQ ID NO: 9.

Aspect 21. The modified AAV particle of aspect 17, wherein the nonhuman primate microdystrophin transgene has a sequence of SEQ ID NO: 10.

Aspect 22. The rAAV particle of aspect 15, wherein the promoter comprises a muscle-specific promoter.

Aspect 23. The rAAV particle of aspect 22, wherein the muscle-specific promoter comprises a human skeletal actin gene element, a cardiac actin gene element, a desmin promoter, a skeletal alpha-actin (ASKA) promoter, a troponin I (TNNI2) promoter, a myocytespecific enhancer binding factor mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor, or any combination thereof.

Aspect 24. A polynucleotide encoding the modified AAVrh74 capsid protein of any one of aspects 1-11.

Aspect 25. The polynucleotide of aspect 24, wherein a sequence of the polynucleotide is SEQ ID NO: 3.

Aspect 26. A plasmid comprising the polynucleotide of aspects 24 or 25.

Aspect 27. A vector system comprising the polynucleotide of aspects 24 or 25, the plasmid of aspect 26, or any combination thereof; wherein the vector system is capable of producing the modified AAV particle of any one of aspects 13-23.

Aspect 28. The vector system of aspect 27, wherein the vector system further comprises a polynucleotide encoding a rep protein or a functional fragment thereof.

Aspect 29. The vector system of aspect 27 or 28, wherein the vector system further comprises a plasmid encoding a transfer cassette.

Aspect 30. The vector system of aspect 29, wherein the transfer cassette comprises a transgene, wherein the transgene comprises a microdystrophin, a dysferlin, an anoctamin-5 transgene, or a fragment thereof.

Aspect 31. A cell comprising the modified AAVrh74 capsid protein of any one of aspects 1-11, the modified AAV particle of any one of aspects 13-23, the polynucleotide of aspects 24 or 25, the plasmid of aspect 26, the vector system of aspect 27-30, or any combination thereof.

Aspect 32. A pharmaceutical composition comprising the modified AAV capsid protein of any one of aspects 1-11, the modified AAV particle of any one of aspects 13-23, or any combination thereof, and a pharmaceutically acceptable carrier.

Aspect 33. The pharmaceutical composition of aspect 32, further comprising 20 mM Tris pH 8, 1 mM MgCl₂, 180-200 mM NaCl, and 0.01%-0.2% poloxamer and optionally 1-3% sucrose.

Aspect 34. The pharmaceutical composition of aspect 32, further comprising 20 mM Tris pH 8, 1 mM MgCl₂, 180-200 mM NaCl, 0.1% poloxamer and 2% sucrose.

Aspect 35. A method of treating a muscular disease comprising administering to a subject in need thereof a therapeutically effective amount of the modified AAV capsid protein of any one of aspects 1-11, the modified AAV particle of any one of aspects 13-23, the pharmaceutical composition of aspects 32-34, or any combination thereof.

Aspect 36. The method of aspect 35, wherein the muscular disease is Duchenne muscular dystrophy, Becker's muscular dystrophy, Myotonic muscular dystrophy, Facioscapulohumeral muscular dystrophy, a limb-girdle muscle dystrophy, or a cardiomyopathy.

Aspect 37. The method of aspect 35 or 36, wherein the subject is a human.

Aspect 38. A composition comprising 20 mM Tris pH 8, 1 mM MgCl₂, 180-200 mM NaCl, and 0.01%-0.2% poloxamer and optionally 1-3% sucrose.

Aspect 39. The composition of aspect 38, further comprising a rAAV vector.

Aspect 40. Use of the modified AAV capsid protein of any one of aspects 1-11, the modified AAV particle of any one of aspects 13-23, the polynucleotide of aspects 24 or 25, the plasmid of aspect 26, the vector system of aspect 27-30, the cell of aspect 31, the composition of aspect 38 or 39, or any combination thereof, in the manufacture of a medicament to treat a muscle disease or disorder in a subject in need thereof.

Aspect 41. The modified AAV capsid protein of any one of aspects 1-11, the modified AAV particle of any one of aspects 13-23, the polynucleotide of aspects 24 or 25, the plasmid of aspect 26, the vector system of aspect 27-30, the cell of aspect 31, the composition of aspect 38 or 39, or any combination thereof, for use in therapy of a muscle disease or disorder.

### BRIEF DESCRIPTION OF THE DRAWINGS

FIGS. 1A-1D provide complement activation (FIG. 1A) and liver enzymes (FIG. 1B=ALT; FIG. 1C-AST; FIG. 1D=total bilirubin) in primates after injection with rh74 or an AAV9-Myo at two doses. ALT=alanine transaminase; AST=aspartate transaminase; LD=low dose; HD=high dose.

FIGS. 2A-2C provide biodistribution, RNA expression, and protein levels in skeletal muscle (FIG. 2A), heart (FIG. 2B), and liver (FIG. 2C) with two doses of AAV9-Myo as compared to the rh74. LD=low dose; HD=high dose.

FIGS. 3A-B Evaluation of different rh74 myotropic variants. Microdystrophin RNA expression at 4 weeks post treatment in skeletal muscle, heart (HRT), diaphragm (DIA), and liver presented as absolute data (FIG. 3A). In a second experiment the data was normalized to the rh74 dose to facilitate more direct comparison (FIG. 3B).

FIGS. 4A-B provides functional outcomes (FIG. 4A=specific force-tibialas anterior; FIG. 4B=eccentric contractions) of treating mice with different viral vectors.

FIGS. 5A-D provide biodistribution in skeletal muscle, heart (FIG. 5A), and liver (FIG. 5B) and expression of the viral vectors and genes (as RNA in FIG. 5C and as percent positive fibers (PFF) for microdystrophin) in the mouse.

FIGS. 6A-E shows complement pathways (FIGS. 6A and 6B) and liver enzymes (FIG. 6C=AST; FIG. 6D=ALT; FIG. 6E=total bilirubin) are elevated in nonhuman primates treated with high- and low-dose MyoAAV (AAV9) but not rh74 or rh74Myo.

### DETAILED DESCRIPTION

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used herein, the terms “increased,” “decreased,” “high,” “low,” or any grammatical variation thereof refer to a variation of about 90%, 80%, 50%, 20%, 10%, 5%, 1%, 0.5%, or even 0.1% of the reference composition, polypeptide, protein, etc.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal sequence identity while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity across the length of the reference sequence and exhibits substantially equivalent biological activity to the reference protein, polypeptide or nucleic acid. Alternatively, when referring to polynucleotides, an equivalent thereof is in one aspect, a polynucleotide that hybridizes under stringent conditions to the reference polynucleotide or its complement that in a further aspect, has the same or similar activity or function as the reference polynucleotide or its complement.

An equivalent of a protein or a polypeptide (referred to herein as the reference) shares at least 50% (or at least 60%, or at least 70%, or at least 80%, or at least 90%) identity to the reference and retains the reference's function and manufacturability.

As used herein, the terms “function,” “activity,” and “enzymatic activity” are used interchangeably.

An equivalent of a polynucleotide (referred to herein as the reference) shares at least 50% (or at least 60%, or at least 70%, or at least 80%, or at least 90%) identity to the reference, and encodes the same polypeptide as the one encoded by the reference, or encodes an equivalent of the polypeptide encoded by the reference.

To arrive at a position or a consecutive segment of a test sequence equivalent to (or corresponding to) an/a amino acid/nucleotide residue or a consecutive segment of a reference sequence, a sequence alignment is performed between the test and reference sequences. The positions or segments aligned to each other are determined as equivalents.

As used herein, the terms “purification,” “purifying,” or “separating” refer to the process of isolating one or more biomaterials (e.g., polynucleotides, polypeptides, or viral vectors) from a complex mixture, such as a cell lysate or a mixture of polypeptides. The purification, separation, or isolation need not be complete, i.e., some components of the complex mixture may remain with the one or more biomaterials (e.g., polynucleotides, polypeptides, or viral vectors) after the purification process. However, the product of purification should be enriched for the one or more biomaterials (e.g., polynucleotides, polypeptides, or viral vectors) relative to the complex mixture before purification and a significant portion of the other components initially present within the complex mixture should be removed by the purification process.

The term “cell” as used herein may refer to either a prokaryotic or eukaryotic cell, optionally obtained from a subject or a commercially available source. In some instances, the cell is a host cell, for example, a mammalian cell or a mammalian host cell. In some instances, the host cell is also referred to herein as a production cell or a packaging cell. In some cases, the cell line is a packaging cell line.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, Chinese Hamster Ovary (CHO) cells, CHO-S cells, CHO-K1 cells, 293T cells, HeLa cells, Baby hamster kidney (BHK) cells, Sf9 cells, yeast cells, stem cells, satellite cells, and muscle cells. Examples of muscle cells include, but are not limited to, skeletal muscle cells, cardiac muscle cells, and smooth muscle cells.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality (for example, having a similar function or activity). It should be understood, without being explicitly stated that when referring to an equivalent or biological equivalent to a reference polypeptide, protein, or polynucleotide, that an equivalent or biological equivalent has the recited structural relationship to the reference polypeptide, protein, or polynucleotide and equivalent or substantially equivalent biological activity. For example, non-limiting examples of equivalent polypeptides, proteins, or polynucleotides include a polypeptide, protein, or polynucleotide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide, polynucleotide or protein sequences across the length of the reference polypeptide, polynucleotide, or protein. Alternatively, an equivalent polypeptide is one that is encoded by a polynucleotide or its complement that hybridizes under conditions of high stringency to a polynucleotide encoding such reference polypeptide sequences and that have substantially equivalent or equivalent biological activity. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity across the length of the reference polynucleotide to the reference polynucleotide, e.g., the wild-type polynucleotide. Such equivalent polypeptides have the same biological activity as the polypeptide encoded by the reference polynucleotide.

Non-limiting examples of equivalent polynucleotides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide. Such equivalent polynucleotides have the same biological activity as the reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences across the length of the reference polynucleotide. The alignment and the percent homology or sequence identity, in one embodiment, can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff-60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address: genome.jp/tools/clustalw/, last accessed on Jan. 13, 2017) or Clustal Omega (available at ebi.ac.uk/Tools/msa/clustalo/). In another embodiment, A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, percent nucleic acid sequence identity is defined as the percentage of nucleotide residues in the candidate sequence that are identical with the nucleotides in the polynucleotide specifically disclosed herein.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, the term “at least 90% identical” refers to an identity of two compared sequences (polynucleotides or polypeptides) of about 90% to about 100%. It also includes an identity of at least at least 91%, at least 92%, at least 93%, at least 94%, at least 95%, at least 96%, at least 97%, at least 98%, at least 99%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

As used herein, the terms “retain,” “similar,” and “same” are used interchangeably while describing a function, an activity or an functional activity of a polynucleotide, a protein and/or a peptide, referring to a functional activity of at least about 20% (including but not limited to: at least about 30%, at least about 40%, at least about 50%, at least about 60%, at least about 70%, at least about 80%, at least about 90%, at least about 95%, at least about 97%, or about 100%) of the activity of the reference protein, polynucleotide and/or peptide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. In one aspect, an equivalent polynucleotide is one that hybridizes under stringent conditions to a reference polynucleotide or its complement. In another aspect, an equivalent polypeptide is a polypeptide that is encoded by a polynucleotide is one that hybridizes under stringent conditions to a reference polynucleotide or its complement.

As used herein, “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, complementary DNA (cDNA), DNA-RNA hybrids, or a polymer comprising purine and pyrimidine bases or other natural, chemically or biochemically modified, non-natural, or derivatized nucleotide bases. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a double-strand (ds) DNA, such as an engineered ds DNA or a ds cDNA synthesized from a single-stranded RNA.

The terms “protein,” “peptide,” and “polypeptide” are used interchangeably and in their broadest sense refer to a compound of two or more subunits of amino acids, amino acid analogs, or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, a consecutive amino acid sequence refers to a sequence having at least two amino acids. However, it is noted that a consecutive amino acid sequence of a first part and a second part does not limit the amino acid sequence to have the first part directly conjugated to the second part. It is also possible that the first part is linked to the second part via a third part, such as a link, thus forming one consecutive amino acid sequence.

As used herein, the terms “conjugate,” “conjugated,” “conjugating,” and “conjugation” refer to the formation of a bond between molecules, and in particular between two amino acid sequences and/or two polypeptides. Conjugation can be direct (i.e., a bond) or indirect (i.e., via a further molecule). The conjugation can be covalent or non-covalent.

As used herein, a consecutive amino acid sequence may comprise two or more polypeptides conjugated with each other directly or indirectly (for example via a linker).

As used herein, the terms “insertion” or “replacement,” with respect to amino acid or nucleotide sequences, refer to differences from the wild-type sequence, rather than the physical act of inserting or replacing a sequence. For example, a peptide described as inserted between amino acids 587 and 591 of a capsid protein (e.g., SEQ ID NO: 4) is positioned between those residues in the sequence. Similarly, a peptide that replaces amino acids 588, 589, and 590 indicates that these residues are absent and the peptide occupies their position in the sequence.

As used herein, the term “recombinant expression system” refers to a genetic construct or constructs for the expression of certain genetic material formed by recombination.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; lipid nanoparticles; polypeptides; polysaccharides; lipopolysaccharides; artificial viral envelopes; metal particles; and bacteria, or viruses, such as rabies virus, flavivirus, lentivirus, baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “mRNA-based delivery,” “transducing,” and the like, as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes, including for example protamine complexes, lipid nanoparticles, polymeric nanoparticles, lipid-polymer hybrid nanoparticles, and inorganic nanoparticles, or combinations thereof) as well as techniques facilitating the delivery of “naked” polynucleotides (such as electroporation, “gene gun” delivery, and various other techniques used for the introduction of polynucleotides). The introduced polynucleotide can be unmodified or can comprise one or more modifications; for example, a modified mRNA may comprise ARCA capping; enzymatic polyadenylation to add a tail of 100-250 adenosine residues; and substitution of one or both of cytidine with 5-methylcytidine and/or uridine with pseudouridine. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors.” Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid.

As used herein, the term “helper” in reference to a virus or plasmid refers to a virus, a nucleic acid sequence, or a plasmid used to provide the additional components necessary for replication and packaging of a viral particle or recombinant viral particle, such as the modified AAV disclosed herein. The components encoded by a helper may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, a “biological sample,” or a “sample,” can be obtained from a subject, cell line, or cultured cell or tissue. Exemplary samples include, but are not limited to, cell sample, tissue sample, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose6 phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly (NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, an epitope tag is a biological structure or sequence, such as a protein or carbohydrate, which acts as an antigen that is recognized by an antibody. In certain embodiments, an epitope tag is used interchangeably with a purification marker and/or an affinity tag.

A “composition” is intended to mean a combination of two or more compounds, such as a combination of an active polypeptide, polynucleotide, viral vector, or antibody and/or another compound or composition, inert (e.g., a detectable label) or active (e.g., a gene delivery vehicle).

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject,” “individual” or “patient” is used interchangeably herein, and refers to a vertebrate, preferably a mammal, more preferably a human. Mammals include, but are not limited to, non-human primates, murines, rats, rabbit, simians, bovines, ovine, porcine, canines, feline, farm animals, sport animals, pets, equine, and primates, particularly human. Besides being useful for human treatment, the present invention is also useful for veterinary treatment of companion mammals, exotic animals and domesticated animals, including mammals, rodents, and the like. In one embodiment, the mammals include horses, dogs, and cats. In another embodiment of the present invention, the human is an adolescent or infant under the age of eighteen years of age.

The term “transduction” or “transduce” refers to viral particle-mediated transfer of genetic material into the cell (e.g., AAV-mediated gene transfer). See, e.g., FIELDS et al., VIROLOGY, volume 2, chapter 69 (3rd ed., Lippincott-Raven Publishers).

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the term “treatment” excludes prevention or prophylaxis.

The term “suffering” as it related to the term “treatment” refers to a patient or individual who has been diagnosed with or is predisposed to a disease.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. In one aspect, an effective amount is a therapeutically effective amount. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to inhibit RNA virus replication ex vivo, in vitro or in vivo.

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

As used herein, the term “AAV” is a standard abbreviation for adeno-associated virus. Natural occurring adeno-associated virus is a single-stranded DNA parvovirus that grows only in cells in which certain functions are provided by a co-infecting helper virus, while recombinant AAV (rAAV) can comprise both a single-stranded DNA or a self-complementary DNA. General information and reviews of AAV can be found in, for example, Carter, Handbook of Parvoviruses 1:169-228, 1989, and Berns, Virology 1743-1764, 1999. However, it is fully expected that these same principles will be applicable to additional AAV serotypes since it is well known that the various serotypes are quite closely related, both structurally and functionally, even at the genetic level. (See, for example, Blacklowe, Parvoviruses and Human Disease 165-174, 1988, J. R. Pattison, ed.; and Rose, Comprehensive Virology 3:1-61, 1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

The term “inverted terminal repeat” or “ITR” includes any palindromic viral terminal repeat or synthetic sequence that forms a hairpin structure and functions as an inverted terminal repeat (i.e., mediates certain viral functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. In one embodiment, the ITR is partially or completely synthetic, such as the “double-D sequence” as described in U.S. Pat. No. 5,478,745 to Samulski et al. See also FIELDS et al., VIROLOGY, volume 2, chapters 69 & 70 (4th ed., Lippincott-Raven Publishers). An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered. An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like.

An “AAV expression cassette,” as used herein, refers to a nucleotide sequence comprising one or more polynucleotides of interest (or transgenes) that are flanked by AAV terminal repeat sequences (ITRs). Such AAV expression cassette can be replicated and packaged into infectious viral particles (e.g., AAV vectors) when present in a host cell that has been transfected with a vector encoding and expressing rep and cap gene products.

In some embodiments, an AAV expression cassette comprises, from 5′ to 3′, a 5′ITR, a promoter and/or an enhancer for expression of the transgene, the transgene itself, a polyA signal sequence, and a 3′ ITR. An expression cassette may also contain an intron. An example of an expression cassette for expression of a microdystrophin is:

(SEQ ID NO: 17)
GCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCC
GCCATGCTACTTATCTACGTAGCCATGCTCTAGAGTTTAAACAAGCTTG
CATGTCTAAGCTAGACCCTTCAGATTAAAAATAACTGAGGTAAGGGCCT
GGGTAGGGGAGGTGGTGTGAGACGCTCCTGTCTCTCCTCTATCTGCCCA
TCGGCCCTTTGGGGAGGAGGAATGTGCCCAAGGACTAAAAAAAGGCCAT
GGAGCCAGAGGGGCGAGGGCAACAGACCTTTCATGGGCAAACCTTGGGG
CCCTGCTGTCTAGCATGCCCCACTACGGGTCTAGGCTGCCCATGTAAGG
AGGCAAGGCCTGGGGACACCCGAGATGCCTGGTTATAATTAACCCAGAC
ATGTGGCTGCCCCCCCCCCCCCAACACCTGCTGCCTCTAAAAATAACCC
TGTCCCTGGTGGATCCCCTGCATGCGAAGATCTTCGAACAAGGCTGTGG
GGGACTGAGGGCAGGCTGTAACAGGCTTGGGGGCCAGGGCTTATACGTG
CCTGGGACTCCCAAAGTATTACTGTTCCATGTTCCCGGCGAAGGGCCAG
CTGTCCCCCGCCAGCTAGACTCAGCACTTAGTTTAGGAACCAGTGAGCA
AGTCAGCCCTTGGGGCAGCCCATACAAGGCCATGGGGCTGGGCAAGCTG
CACGCCTGGGTCCGGGGTGGGCACGGTGCCCGGGCAACGAGCTGAAAGC
TCATCTGCTCTCAGGGGCCCCTCCCTGGGGACAGCCCCTCCTGGCTAGT
CACACCCTGTAGGCTCCTCTATATAACCCAGGGGCACAGGGGCTGCCCT
CATTCTACCACCACCTCCACAGCACAGACAGACACTCAGGAGCAGCCAG
CGGCGCGCCCAGGTAAGTTTAGTCTTTTTGTCTTTTATTTCAGGTCCCG
GATCCGGTGGTGGTGCAAATCAAAGAACTGCTCCTCAGTGGATGTTGCC
TTTACTTCTAGGCCTGTACGGAAGTGTTACTTCTGCTCTAAAAGCTGCG
GAATTGTACCCGCGGCCGCCACCATGCTGTGGTGGGAGGAGGTGGAGGA
TTGTTATGAAAGGGAGGACGTGCAGAAGAAGACTTTTACCAAGTGGGTG
AACGCTCAGTTCAGCAAATTTGGGAAGCAGCACATCGAGAATCTGTTTT
CCGACCTGCAGGATGGGAGACGGCTGCTGGATCTGCTGGAAGGACTGAC
TGGCCAGAAGCTGCCCAAAGAGAAGGGGAGCACTAGGGTGCACGCCCTG
AACAACGTGAACAAAGCTCTGAGAGTGCTGCAGAACAACAACGTGGATC
TGGTGAATATTGGCAGTACTGATATCGTGGACGGGAACCACAAACTGAC
ACTGGGCCTGATCTGGAACATTATTCTGCACTGGCAGGTGAAAAATGTG
ATGAAGAACATCATGGCCGGGCTGCAGCAGACCAATTCCGAGAAGATCC
TGCTGTCTTGGGTGCGGCAGAGCACCCGCAACTATCCCCAGGTGAACGT
GATTAACTTCACTACATCCTGGAGCGACGGGCTGGCCCTGAATGCTCTG
ATTCACAGCCACAGGCCTGATCTGTTCGACTGGAATAGCGTGGTGTGCC
AGCAGTCTGCCACACAGCGCCTGGAACATGCCTTCAATATCGCTCGGTA
CCAGCTGGGGATCGAAAAACTGCTGGACCCAGAGGATGTGGACACTACA
TACCCAGATAAAAAGTCTATTCTGATGTACATTACTAGCCTGTTCCAGG
TGCTGCCACAGCAGGTGTCTATTGAAGCCATTCAGGAGGTGGAAATGCT
GCCCCGCCCCCCCAAAGTGACTAAAGAGGAGCATTTTCAGCTGCATCAT
CAGATGCATTACAGCCAGCAGATTACCGTGAGCCTGGCTCAGGGATATG
AGCGCACCAGTAGTCCAAAACCACGGTTCAAGTCCTACGCTTATACCCA
GGCTGCCTACGTGACAACTAGCGACCCTACTAGATCCCCCTTTCCATCC
CAGCACCTGGAGGCCCCAGAGGACAAGAGCTTTGGGTCCAGCCTGATGG
AAAGCGAGGTGAATCTGGATCGGTACCAGACAGCCCTGGAGGAGGTGCT
GAGCTGGCTGCTGAGTGCTGAAGACACACTGCAGGCCCAGGGCGAAATT
TCCAATGACGTGGAAGTGGTGAAGGATCAGTTCCACACACACGAGGGCT
ATATGATGGACCTGACAGCTCACCAGGGGCGCGTGGGCAATATCCTGCA
GCTGGGCTCTAAACTGATCGGCACCGGGAAACTGAGTGAGGACGAGGAA
ACAGAAGTGCAGGAGCAGATGAACCTGCTGAACAGCCGCTGGGAGTGTC
TGAGAGTGGCTAGTATGGAGAAGCAGTCCAACCTGCACCGGGTGCTGAT
GGACCTGCAGAACCAGAAACTGAAAGAGCTGAACGACTGGCTGACAAAG
ACTGAGGAACGCACAAGGAAGATGGAGGAGGAGCCACTGGGACCCGACC
TGGAGGATCTGAAGAGACAGGTGCAGCAGCATAAGGTGCTGCAGGAGGA
TCTGGAACAGGAGCAGGTGCGGGTGAACTCCCTGACACATATGGTGGTG
GTGGTGGACGAATCTAGTGGAGATCACGCCACCGCCGCCCTGGAGGAAC
AGCTGAAGGTGCTGGGGGACCGGTGGGCCAACATTTGCCGGTGGACCGA
GGACAGGTGGGTGCTGCTGCAGGACATCCTGCTGAAATGGCAGAGGCTG
ACCGAGGAGCAGTGTCTGTTTAGTGCTTGGCTGAGCGAGAAAGAGGACG
CCGTGAACAAGATCCACACAACCGGCTTTAAGGATCAGAACGAAATGCT
GTCTAGCCTGCAGAAACTGGCTGTGCTGAAGGCCGATCTGGAGAAAAAG
AAGCAGAGCATGGGCAAACTGTATAGCCTGAAACAGGACCTGCTGAGCA
CCCTGAAGAACAAGAGCGTGACCCAGAAGACAGAAGCCTGGCTGGATAA
CTTTGCCCGCTGCTGGGACAACCTGGTGCAGAAACTGGAGAAAAGTACA
GCTCAGATCTCTCAGGCTGTGACCACAACCCAGCCTAGCCTGACCCAGA
CAACCGTGATGGAAACCGTGACCACCGTGACAACCCGCGAACAGATCCT
GGTGAAACATGCCCAGGAAGAGCTGCCACCTCCACCTCCCCAGAAGAAG
AGAACCCTGGAGCGGCTGCAGGAGCTGCAGGAAGCCACTGACGAACTGG
ACCTGAAGCTGAGGCAGGCCGAAGTGATTAAGGGGTCTTGGCAGCCTGT
GGGCGATCTGCTGATTGATTCCCTGCAGGACCACCTGGAAAAGGTGAAG
GCTCTGAGAGGCGAAATTGCTCCACTGAAGGAGAACGTGAGTCATGTGA
ACGATCTGGCTAGACAGCTGACAACACTGGGCATCCAGCTGAGCCCATA
CAATCTGAGCACACTGGAGGACCTGAATACCAGGTGGAAGCTGCTGCAG
GTGGCTGTGGAAGACCGGGTGCGGCAGCTGCATGAGGCCCATCGCGACT
TCGGACCAGCCAGCCAGCACTTTCTGAGCACATCCGTGCAGGGGCCCTG
GGAGAGGGCCATTTCTCCCAACAAGGTGCCCTACTATATTAATCACGAG
ACCCAGACCACTTGTTGGGACCATCCCAAGATGACAGAACTGTACCAGT
CCCTGGCCGATCTGAACAACGTGAGGTTTAGCGCTTACAGAACCGCTAT
GAAGCTGAGACGGCTGCAGAAGGCCCTGTGCCTGGATCTGCTGTCCCTG
TCCGCCGCCTGCGATGCCCTGGATCAGCATAATCTGAAGCAGAACGATC
AGCCAATGGATATCCTGCAGATCATCAACTGCCTGACCACTATCTACGA
CAGGCTGGAGCAGGAGCACAACAACCTGGTGAACGTGCCTCTGTGCGTG
GATATGTGCCTGAACTGGCTGCTGAACGTGTATGACACTGGGCGCACCG
GCCGGATCAGAGTGCTGAGTTTTAAAACTGGGATTATCTCCCTGTGTAA
GGCCCACCTGGAGGACAAGTACAGGTACCTGTTCAAGCAGGTGGCTAGT
AGCACTGGATTTTGTGACCAGCGCCGCCTGGGACTGCTGCTGCATGATA
GTATCCAGATTCCTAGACAGCTGGGAGAGGTGGCTAGTTTCGGAGGATC
TAACATCGAACCCAGCGTGCGCAGCTGTTTCCAGTTTGCCAATAACAAA
CCTGAAATCGAGGCTGCTCTGTTCCTGGATTGGATGCGCCTGGAACCAC
AGAGCATGGTGTGGCTGCCTGTGCTGCACAGAGTGGCTGCCGCCGAAAC
TGCCAAGCACCAGGCTAAATGCAACATCTGCAAGGAATGTCCCATTATC
GGCTTTCGCTACAGGAGTCTGAAACATTTTAACTACGATATTTGCCAGA
GCTGCTTCTTTTCCGGAAGAGTGGCCAAAGGACACAAGATGCACTACCC
TATGGTGGAATATTGCACCCCAACTACATCTGGCGAAGATGTGCGCGAT
TTTGCCAAGGTGCTGAAGAATAAGTTTCGGACTAAGAGGTACTTCGCCA
AGCACCCCCGCATGGGGTATCTGCCAGTGCAGACAGTGCTGGAAGGAGA
CAATATGGAGACCGATACAATGTGAGCGGCCGCAATAAAAGATCTTTAT
TTTCATTAGATCTGTGTGTTGGTTTTTTGTGTGTCTAGAGTCGACCAGA
GCATGGCTACGTAGATAAGTAGCATGGCGGGTTAATCATTAACTACAAG
GAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCG
GGCGGCCTCAGTGAGCGAGCGAGCGCGC.

An “AAV virion” or “AAV vector” or “AAV viral particle” or “AAV vector particle” refers to a viral particle composed of at least one AAV capsid protein and an encapsidated polynucleotide AAV expression cassette. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV expression cassette, as such a nucleotide is contained within an AAV vector particle.

Natural occurring adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983) as corrected by Ruffing et al., J Gen Virol, 75:3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). Cloning of the AAVrh.74 serotype is described in Rodino-Klapac. et al. Journal of Translational Medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. All VPs share the same C-terminus amino acid sequence. VP2 has an additional N-terminal portion compared to VP3, and VP1 has an additional N-term portion compared to VP2 (and VP3). A complete AAV capsid comprises 60 subunits, with polypeptides VP1:VP2:VP3 in an approximate ratio of 1:1:10. A single consensus polyadenylation site is located at map position 95 of the AAV genome. The life cycle and genetics of AAV are reviewed in Muzyczka, Current Topics in Microbiology and Immunology, 158:97-129 (1992).

Recombinant AAV genomes of the disclosure comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAVrh. 10, AAVrh.20, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). As noted in the Background section above, the nucleotide sequences of the genomes of various AAV serotypes are known in the art. In some embodiments, to promote skeletal muscle specific expression, the AAVrh.74 capsid gene is used.

As used herein, the term “micro-dystrophin” refers to a truncated dystrophin protein comprising four or fewer spectrin repeats. Non-limiting examples of mini- and micro-dystrophins are: DysΔR4-R23/ΔCTD (delandistrogene moxeparvovec, μDysH2), DysΔ17-48, DysΔH2-R19, DysΔH2-R15, DysΔR2-23, ΔR2-15/ΔR18-22/ΔCTD (μDys5), ΔR3-19/ΔR20-21/ΔCTD, ΔR2-15/ΔR18-19/ΔR20-23/ΔCTD, Dys3978, Dys3849, minidystrophin d3990, RGX-202, mDys5R (SGT-001), and DYSF. Examples of microdystrophin sequences may be found (without limitation) in WO2023/018854, WO2015/197869, WO2023/015304, and WO2021/108755.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated,” as used herein, with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “recombinant” as used herein with respect to polypeptides or polynucleotides, such as DNA or RNA, refers to molecules formed by laboratory methods of recombination, such as molecular cloning. Molecular cloning techniques are known in the art and may include, but is not limited to, PCR amplification of a polynucleotide, enzymatic digestion of a polynucleotide, ligation of a polynucleotide into an expression cassette (e.g., mammalian expression cassette), transformation, transfection or transduction of a cell with the polynucleotide, and expression of the polynucleotide to produce the polypeptide. See e.g., Green and Sambrook, Molecular Cloning: A Laboratory Manual, 2012. The term “recombinant polynucleotide” is meant to include fragments of protein-encoding polynucleotides. For instance, a recombinant polynucleotide may include a fragment of the polynucleotide that encodes for a human dysferlin protein. A recombinant polynucleotide may be produced by PCR amplification of a fragment of a protein-encoding polynucleotide. A recombinant polypeptide may be produced by expression of one or more recombinant polynucleotides. In one embodiment, the “recombinant” polypeptide means a polypeptide that is separated or substantially free from at least some of the other components of the naturally occurring organism or virus, for example, the cell or viral structural components or other polypeptides or nucleic acids commonly found associated with the polypeptide.

The term “Rep sequence” or “Rep coding sequence” means the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers). The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40); in fact, it is believed that AAVS only express the spliced Rep68 and Rep40 proteins. In representative embodiments, the Rep coding sequences encode at least those replication proteins that are necessary for viral or vector genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins. In the native AAV genome, the different Rep proteins are encoded by a single gene through use of two different promoters and alternative splicing. For purposes of AAV vector production, however, Rep proteins can be expressed in producer cells from a single gene, or from distinct polynucleotides, one sequence for each Rep protein to be expressed. Thus, for example, a Rep encoding gene can be engineered to inactivate the p5 or p19 promoter so that only small or only large Rep proteins are expressed the respective modified genes.

As used herein, the term “cap sequence” or “cap coding sequences” refers to the structural proteins that form a functional capsid (i.e., can package DNA and infect target cells), e.g., parvovirus or AAV capsids. In one embodiment, the cap coding sequences encode all of the parvovirus or AAV capsid subunits, but less than all of the capsid subunits may be encoded as long as a functional capsid is produced. In another embodiment, the cap sequences are present on a single nucleic acid molecule. The capsid structure of autonomous parvoviruses and AAV are described in more detail in BERNARD N. FIELDS et al., VIROLOGY, volume 2, chapters 69 & 70 (4th ed., Lippincott-Raven Publishers).

Disclosed herein are capsid sequences modified by the insertion of myotropic sequences. AAV capsids can accommodate insertions, but the tolerability of the capsid for the insertion varies greatly with the insertion sequence and the insertion position, both in terms of the surrounding capsid sequence and of the capsid secondary structure. (Hoffman, Mol. Therapy 31:1-21 (2023)).

Disclosed herein are polynucleotides encoding a protein of interest or its fragment. Further disclosed herein are plasmids, viral vectors, vector systems, viral packaging systems, cells, and compositions comprising polynucleotides encoding a protein of interest or its fragment. Also disclosed herein are methods of making and using such polynucleotides, plasmids, viral vectors, vector systems, viral packaging systems, cells, and compositions.

In some embodiments, an adeno-associated viral (AAV) vector comprises: (a) a first inverted terminal repeat (ITR); (b) a polynucleotide encoding a gene of interest; and (c) a second ITR, wherein the polynucleotide is flanked by the first and second ITRs.

Further disclosed herein are adeno-associated viral (AAV) expression cassettes. In some embodiments, the AAV expression cassette comprises: (a) a first inverted terminal repeat (ITR), wherein the first ITR comprises any of the ITRs disclosed herein; (b) a polynucleotide encoding a gene of interest; and (c) a second ITR, wherein the second ITR comprises any of the ITRs disclosed herein, wherein the polynucleotide encoding the gene of interest (b) is flanked by the first and second ITRs of (a) and (c).

In some embodiments, the polynucleotides, plasmids, viral vectors (e.g., viruses or viral particles), vector systems, viral packaging systems, cells, and compositions further comprise one or more nucleotide sequences comprising, consisting of, or consisting essentially of an inverted terminal repeat (ITR), promoter, intron, selection marker, a therapeutic gene, or origin of replication (ORI).

In some embodiments, the polynucleotides, plasmids, viral vectors, vector systems, viral packaging systems, cells, and compositions further comprise one or more additional nucleotide sequences comprising an inverted terminal repeat (ITR), selection marker, origin of replication (ORI), untranslated region (UTR), or polyadenylation (polyA) signal.

Further disclosed herein are methods of treating a muscle disease or disorder. In some embodiments, a method of treating the muscle disease or disorder comprises administering to a subject in need thereof any of polynucleotides, plasmids, viral vectors, vector systems, viral packaging systems, cells, and compositions disclosed herein.

Further disclosed herein are uses of any of the polynucleotides, plasmids, viral vectors, vector systems, viral packaging systems, cells, and compositions disclosed herein in the manufacture of a medicament for the treatment of a muscle disease or disorder.

In some embodiments, the polynucleotide of the invention is operably linked to a polyadenylation element. In some embodiments, the polynucleotide is part of an expression cassette comprising, consisting essentially of, or consisting or the polynucleotide operably linked to a promoter and a polyadenylation element.

In certain embodiments, the viral vector exhibits modified tissue tropism compared to vectors from which the modified vector is derived. In one embodiment, the parvovirus vector exhibits systemic tropism for skeletal, cardiac, and/or diaphragm muscle. In other embodiments, the parvovirus vector has reduced tropism for liver compared to a virus vector comprising a wild-type capsid protein. Tissue tropism can be modified by altering certain viral capsid amino acids, for example, those present in AAVcapsid VP1, VP2, and/or VP3 proteins, according to the knowledge of those ordinarily skilled in the art.

In some embodiments, the vector genome is self-complementary or duplexed, and AAV virions containing such vector genomes are known as scAAV vectors. scAAV vectors are described in international patent publication WO 01/92551 (the disclosure of which is incorporated herein by reference in its entirety). Use of scAAV to express a mini-dystrophin may provide an increase in the number of cells transduced, the copy number pertransduced cell, or both.

An additional aspect of the invention relates to a transfected, transduced or transformed cell comprising the polynucleotide and/or vector of the invention. The cell may be an in vitro, ex vivo, or in vivo cell. A further aspect of the invention relates to a non-human transgenic animal comprising the polynucleotide and/or vector and/or transformed cell of the invention. In some embodiments, the transgenic animal is a laboratory animal, e.g., an animal model of a disease, e.g., an animal model of muscular dystrophy.

### Microdystrophin

An additional aspect of the invention relates to a method of treating muscular dystrophy in a subject in need thereof, comprising delivering to the subject a therapeutically effective amount of the polynucleotide, vector, and/or transformed cell of the invention, thereby treating muscular dystrophy in the subject. The muscular dystrophy may be any form of muscular dystrophy, e.g., Duchenne muscular dystrophy, Becker muscular dystrophy, or any of the various types of Limb Girdle Muscular Dystrophy. An additional aspect of the invention relates to a method of treating a cardiac disorder in a subject in need thereof, comprising delivering to the subject a therapeutically effective amount of the polynucleotide, vector, and/or transformed cell of the invention, thereby treating the cardiac disorder in the subject. (Argiro, A. et al., JACC Heart Fail. 12 (2): 248-260 (2023)).

Gene therapies for the treatment of DMD comprise administering to the subjects a gene therapy vector (e.g., an AAV gene therapy vector) comprising a vector genome encoding one or more portions of a functional Dystrophin protein, (e.g., a mini- or micro-Dystrophin). Non-limiting example of mini- or micro-Dystrophins that can be encoded by gene therapy vectors for the treatment of DMD are DysΔR4-R23/ΔCTD (delandistrogene moxeparvovec, μDysH2), DysΔ17-48, DysΔH2-R19, DysΔH2-R15, DysΔR2-23, ΔR2-15/ΔR18-22/ΔCTD (μDys5), ΔR3-19/ΔR20-21/ΔCTD, ΔR2-15/ΔR18-19/ΔR20-23/ΔCTD, Dys3978, Dys3849, minidystrophin d3990, RGX-202, mDys5R (SGT-001), and DYSF. A vector genome comprised in a gene therapy vector (e.g., an AAV gene therapy vector) for the treatment of DMD can comprise one or more portions of a Dystrophin gene encoding a functional Dystrophin protein, or a functional portion thereof (e.g., a mini- or micro-Dystrophin). In some aspects, the micro-Dystrophin encoded by gene therapy vectors for the treatment of DMD is DysΔR4-R23/ΔCTD (delandistrogene moxeparvovec, μDysH2). In some aspects, the micro-Dystrophin encoded by gene therapy vectors for the treatment of DMD is delandistrogene moxeparvovec dystrophin.

In some aspects, a vector genome comprised in a gene therapy vector (e.g., AAV gene therapy vectors) for the treatment of DMD can comprise one or more exons (or one or more portions thereof) of a Dystrophin gene encoding a functional Dystrophin protein, or a functional portion thereof (e.g., a mini- or micro-Dystrophin). In some aspects, a vector genome comprised in a gene therapy vector (e.g., an AAV gene therapy vector) for the treatment of DMD can comprise one or more portions of any one of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof, of a Dystrophin gene. In some aspects, a vector genome comprised in a gene therapy vector (e.g., a AAV gene therapy vector) for the treatment of DMD can comprise one or more of any one of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof, of a Dystrophin gene.

In some aspects, a vector genome comprised in a gene therapy vector (e.g., a AAV gene therapy vector) for the treatment of DMD can comprise one or more portions of any one of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene. In some aspects, a vector genome comprised in a gene therapy vector (e.g., a AAV gene therapy vector) for the treatment of DMD can comprise one or more of any one of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene.

In some aspects, a vector genome comprised in a gene therapy vector (e.g., an AAV gene therapy vector) for the treatment of DMD can encode one or more portions of a functional Dystrophin protein, or a functional portion thereof (e.g., a mini- or micro-Dystrophin). In some aspects, a vector genome comprised in a gene therapy vector (e.g., a AAV gene therapy vector) for the treatment of DMD can encode one or more portions of a dystrophin protein acting binding domain, a dystrophin protein spectrin-like repeat 1, a dystrophin protein spectrin-like repeat 2, a dystrophin protein spectrin-like repeat 3, a dystrophin protein spectrin-like repeat 4, a dystrophin protein spectrin-like repeat 5, a dystrophin protein spectrin-like repeat 6, a dystrophin protein spectrin-like repeat 7, a dystrophin protein spectrin-like repeat 8, a dystrophin protein spectrin-like repeat 9, a dystrophin protein spectrin-like repeat 10, a dystrophin protein spectrin-like repeat 11, a dystrophin protein spectrin-like repeat 12, a dystrophin protein spectrin-like repeat 13, a dystrophin protein spectrin-like repeat 14, a dystrophin protein spectrin-like repeat 15, a dystrophin protein spectrin-like repeat 16, a dystrophin protein spectrin-like repeat 17, a dystrophin protein spectrin-like repeat 18, a dystrophin protein spectrin-like repeat 19, a dystrophin protein spectrin-like repeat 20, a dystrophin protein spectrin-like repeat 21, a dystrophin protein spectrin-like repeat 22, a dystrophin protein spectrin-like repeat 23, a dystrophin protein spectrin-like repeat 24, a dystrophin protein hinge region 1, a dystrophin protein hinge region 2, a dystrophin protein hinge region 3, or a dystrophin protein hinge region 4, a dystrophin protein cysteine-rich domain, a dystrophin protein carboxyl-terminus, or any combination thereof. In some aspects, a vector genome comprised in a gene therapy vector (e.g., an AAV gene therapy vector) for the treatment of DMD can encode one or more of a dystrophin protein acting binding domain, a dystrophin protein spectrin-like repeat 1, a dystrophin protein spectrin-like repeat 2, a dystrophin protein spectrin-like repeat 3, a dystrophin protein spectrin-like repeat 4, a dystrophin protein spectrin-like repeat 5, a dystrophin protein spectrin-like repeat 6, a dystrophin protein spectrin-like repeat 7, a dystrophin protein spectrin-like repeat 8, a dystrophin protein spectrin-like repeat 9, a dystrophin protein spectrin-like repeat 10, a dystrophin protein spectrin-like repeat 11, a dystrophin protein spectrin-like repeat 12, a dystrophin protein spectrin-like repeat 13, a dystrophin protein spectrin-like repeat 14, a dystrophin protein spectrin-like repeat 15, a dystrophin protein spectrin-like repeat 16, a dystrophin protein spectrin-like repeat 17, a dystrophin protein spectrin-like repeat 18, a dystrophin protein spectrin-like repeat 19, a dystrophin protein spectrin-like repeat 20, a dystrophin protein spectrin-like repeat 21, a dystrophin protein spectrin-like repeat 22, a dystrophin protein spectrin-like repeat 23, a dystrophin protein spectrin-like repeat 24, a dystrophin protein hinge region 1, a dystrophin protein hinge region 2, a dystrophin protein hinge region 3, or a dystrophin protein hinge region 4, a dystrophin protein cysteine-rich domain, a dystrophin protein carboxyl-terminus, or any combination thereof.

In some aspects, a vector genome comprised in a gene therapy vector (e.g., a AAV gene therapy vector) for the treatment of DMD can encode one or more portions of a dystrophin protein acting binding domain, a dystrophin protein hinge region 1, a dystrophin protein spectrin-like repeat 1, a dystrophin protein spectrin-like repeat 2, a dystrophin protein spectrin-like repeat 3, a dystrophin protein hinge region 2, a dystrophin protein spectrin-like repeat 24, dystrophin protein hinge region 4, a dystrophin protein carboxyl-terminus, or any combination thereof. In some aspects, a vector genome comprised in a gene therapy vector (e.g., a AAV gene therapy vector) for the treatment of DMD can encode one or more of a dystrophin protein acting binding domain, a dystrophin protein hinge region 1, a dystrophin protein spectrin-like repeat 1, a dystrophin protein spectrin-like repeat 2, a dystrophin protein spectrin-like repeat 3, a dystrophin protein hinge region 2, a dystrophin protein spectrin-like repeat 24, dystrophin protein hinge region 4, a dystrophin protein carboxyl-terminus, or any combination thereof.

In some aspects, the genome of the subject suffering from DMD comprises one or more defective copies (i.e., comprising a mutation) of a Dystrophin gene encoding a non-functional or sub-functional (i.e., having reduced function compared to a Dystrophin protein encoded by a gene not comprising the mutation) Dystrophin protein. In some aspects, the Dystrophin gene comprised in the subject's genome lacks one or more portions of a Dystrophin gene. In some aspects, the Dystrophin gene comprised in the subject's genome lacks one or more portions of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof. In some aspects, the Dystrophin gene comprised in the subject's genome lacks one or more of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof.

In some aspects, the Dystrophin gene comprised in the subject's genome lacks one or more portions of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene. In some aspects, the Dystrophin gene comprised in the subject's genome lacks one or more of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene.

In some aspects, the Dystrophin gene comprised in the subject's genome comprises but does not express (e.g., due to the presence of a mutation) one or more portions of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof. In some aspects, the Dystrophin gene comprised in the subject's genome comprises but does not express (e.g., due to the presence of a mutation) one or more of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71, exon 72, exon 73, exon 74, exon 75, exon 76, exon 77, exon 78, exon 79, or any combination thereof.

In some aspects, the Dystrophin gene comprised in the subject's genome comprises but does not express (e.g., due to the presence of a mutation) one or more portions of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene. In some aspects, the Dystrophin gene comprised in the subject's genome comprises but does not express (e.g., due to the presence of a mutation) one or more of exon 1, exon 2, exon 3, exon 4, exon 5, exon 6, exon 7, exon 8, exon 9, exon 10, exon 11, exon 12, exon 13, exon 14, exon 15, exon 16, exon 17, exon 59, exon 60, exon 61, exon 62, exon 63, exon 64, exon 65, exon 66, exon 67, exon 68, exon 69, or any combination thereof, of a Dystrophin gene.

In some aspects, the non-functional or sub-functional (i.e., having reduced function compared to a Dystrophin protein encoded by a gene not comprising the mutation) Dystrophin protein encoded by the Dystrophin gene(s) comprised in the subject's genome lacks one or more portion of a Dystrophin protein. In some aspects, the non-functional or sub-functional (i.e., having reduced function compared to a Dystrophin protein encoded by a gene not comprising the mutation) Dystrophin protein encoded by the Dystrophin gene(s) comprised in the subject's genome lacks one or more portion of a dystrophin protein acting binding domain, a dystrophin protein spectrin-like repeat 1, a dystrophin protein spectrin-like repeat 2, a dystrophin protein spectrin-like repeat 3, a dystrophin protein spectrin-like repeat 4, a dystrophin protein spectrin-like repeat 5, a dystrophin protein spectrin-like repeat 6, a dystrophin protein spectrin-like repeat 7, a dystrophin protein spectrin-like repeat 8, a dystrophin protein spectrin-like repeat 9, a dystrophin protein spectrin-like repeat 10, a dystrophin protein spectrin-like repeat 11, a dystrophin protein spectrin-like repeat 12, a dystrophin protein spectrin-like repeat 13, a dystrophin protein spectrin-like repeat 14, a dystrophin protein spectrin-like repeat 15, a dystrophin protein spectrin-like repeat 16, a dystrophin protein spectrin-like repeat 17, a dystrophin protein spectrin-like repeat 18, a dystrophin protein spectrin-like repeat 19, a dystrophin protein spectrin-like repeat 20, a dystrophin protein spectrin-like repeat 21, a dystrophin protein spectrin-like repeat 22, a dystrophin protein spectrin-like repeat 23, a dystrophin protein spectrin-like repeat 24, a dystrophin protein hinge region 1, a dystrophin protein hinge region 2, a dystrophin protein hinge region 3, or a dystrophin protein hinge region 4, a dystrophin protein cysteine-rich domain, a dystrophin protein carboxyl-terminus, or any combination thereof. In some aspects, the non-functional or sub-functional (i.e., having reduced function compared to a Dystrophin protein encoded by a gene not comprising the mutation) Dystrophin protein encoded by the Dystrophin gene(s) comprised in the subject's genome lacks one or more of a dystrophin protein acting binding domain, a dystrophin protein spectrin-like repeat 1, a dystrophin protein spectrin-like repeat 2, a dystrophin protein spectrin-like repeat 3, a dystrophin protein spectrin-like repeat 4, a dystrophin protein spectrin-like repeat 5, a dystrophin protein spectrin-like repeat 6, a dystrophin protein spectrin-like repeat 7, a dystrophin protein spectrin-like repeat 8, a dystrophin protein spectrin-like repeat 9, a dystrophin protein spectrin-like repeat 10, a dystrophin protein spectrin-like repeat 11, a dystrophin protein spectrin-like repeat 12, a dystrophin protein spectrin-like repeat 13, a dystrophin protein spectrin-like repeat 14, a dystrophin protein spectrin-like repeat 15, a dystrophin protein spectrin-like repeat 16, a dystrophin protein spectrin-like repeat 17, a dystrophin protein spectrin-like repeat 18, a dystrophin protein spectrin-like repeat 19, a dystrophin protein spectrin-like repeat 20, a dystrophin protein spectrin-like repeat 21, a dystrophin protein spectrin-like repeat 22, a dystrophin protein spectrin-like repeat 23, a dystrophin protein spectrin-like repeat 24, a dystrophin protein hinge region 1, a dystrophin protein hinge region 2, a dystrophin protein hinge region 3, or a dystrophin protein hinge region 4, a dystrophin protein cysteine-rich domain, a dystrophin protein carboxyl-terminus, or any combination thereof.

In some aspects, the non-functional or sub-functional (i.e., having reduced function compared to a Dystrophin protein encoded by a gene not comprising the mutation) Dystrophin protein encoded by the Dystrophin gene(s) comprised in the subject's genome lacks one or more portion of a dystrophin protein acting binding domain, a dystrophin protein hinge region 1, a dystrophin protein spectrin-like repeat 1, a dystrophin protein spectrin-like repeat 2, a dystrophin protein spectrin-like repeat 3, a dystrophin protein hinge region 2, a dystrophin protein spectrin-like repeat 24, dystrophin protein hinge region 4, a dystrophin protein carboxyl-terminus, or any combination thereof. In some aspects, the non-functional or sub-functional (i.e., having reduced function compared to a Dystrophin protein encoded by a gene not comprising the mutation) Dystrophin protein encoded by the Dystrophin gene(s) comprised in the subject's genome lacks one or more of a dystrophin protein acting binding domain, a dystrophin protein hinge region 1, a dystrophin protein spectrin-like repeat 1, a dystrophin protein spectrin-like repeat 2, a dystrophin protein spectrin-like repeat 3, a dystrophin protein hinge region 2, a dystrophin protein spectrin-like repeat 24, dystrophin protein hinge region 4, a dystrophin protein carboxyl-terminus, or any combination thereof.

In some aspects, a vector genome comprised in a gene therapy vector (e.g., AAV gene therapy vectors) for the treatment of DMD comprises one or more portions of exon 8 and/or exon 9 of a Dystrophin gene encoding a functional Dystrophin protein. In some aspects, a vector genome comprised in a gene therapy vector (e.g., AAV gene therapy vectors) for the treatment of DMD encodes a micro-Dystrophin protein comprising: a dystrophin protein acting binding domain; dystrophin protein spectrin-like repeats 1, 2, 3, and 24; dystrophin protein hinge regions 1, 2, and 4; and a dystrophin protein cysteine-rich domain, or any combination thereof. In some aspects, a vector genome comprised in a gene therapy vector (e.g., AAV gene therapy vectors) for the treatment of DMD encodes a micro-Dystrophin protein comprising a dystrophin protein hinge region 1. In some aspects, a vector genome comprised in a gene therapy vector (e.g., AAV gene therapy vectors) for the treatment of DMD encodes a DysΔR4-R23/ΔCTD micro-Dystrophin protein. In some aspects, a vector genome comprised in a gene therapy vector (e.g., AAV gene therapy vectors) for the treatment of DMD encodes a μDysH2 micro-Dystrophin protein. In some aspects, the gene therapy is delandistrogene moxeparvovec dystrophin gene therapy.

In some aspects, the Dystrophin gene(s) comprised in the genome of the subject suffering from DMD, to whom the gene therapy vector (e.g., AAV gene therapy vectors) for the treatment of DMD is administered, lacks one or more portions of exon 8 and/or exon 9 of a Dystrophin gene. In some aspects, the Dystrophin gene(s) comprised in the genome of the subject suffering from DMD, to whom the gene therapy vector (e.g., AAV gene therapy vectors) for the treatment of DMD is administered, lacks exon 8 and/or exon 9 of a Dystrophin gene. In some aspects, the genome of the subjects suffering from DMD, to whom the gene therapy vector (e.g., AAV gene therapy vectors) for the treatment of DMD is administered, encodes a Dystrophin protein lacking one or more portions of a dystrophin protein acting binding domain; dystrophin protein spectrin-like repeats 1, 2, 3, and 24; dystrophin protein hinge regions 1, 2, and 4; and a dystrophin protein cysteine-rich domain, or any combination thereof. In some aspects, the genome of the subjects suffering from DMD, to whom the gene therapy vector (e.g., AAV gene therapy vectors) for the treatment of DMD is administered, encodes a Dystrophin protein lacking one or more of a dystrophin protein acting binding domain; dystrophin protein spectrin-like repeats 1, 2, 3, and 24; dystrophin protein hinge regions 1, 2, and 4; and a dystrophin protein cysteine-rich domain, or any combination thereof. In some aspects, the genome of the subjects suffering from DMD, to whom the gene therapy vector (e.g., AAV gene therapy vectors) for the treatment of DMD is administered, encodes a Dystrophin protein lacking one or more portions of a dystrophin protein hinge region 1. In some aspects, the genome of the subjects suffering from DMD, to whom the gene therapy vector (e.g., AAV gene therapy vectors) for the treatment of DMD is administered, encodes a Dystrophin protein lacking a dystrophin protein hinge region 1.

In some aspects, a Dystrophin gene comprised in the genome of the subject suffering from DMD, to whom a gene therapy vector (e.g., AAV gene therapy vectors) for the treatment of DMD is administered, comprises one or more portions of one or more exons (or portions thereof) of a Dystrophin gene comprised in the administered gene therapy vector (e.g., AAV gene therapy vectors) for the treatment of DMD.

In some aspects, a Dystrophin gene comprised in the genome of the subject suffering from DMD, to whom a gene therapy vector (e.g., AAV gene therapy vectors) for the treatment of DMD is administered, comprises one or more exons of a Dystrophin gene comprised in the administered gene therapy vector (e.g., AAV gene therapy vectors) for the treatment of DMD.

In some aspects, a Dystrophin gene comprised in the genome of the subject suffering from DMD, to whom a gene therapy vector (e.g., AAV gene therapy vectors) for the treatment of DMD is administered, comprises all the exons (or portions thereof) of a Dystrophin gene comprised in the administered gene therapy vector (e.g., AAV gene therapy vectors) for the treatment of DMD.

In some aspects, the Dystrophin gene(s) comprised in the genome of the subject suffering from DMD, to whom the gene therapy vector (e.g., AAV gene therapy vectors) for the treatment of DMD is administered, lacks one or more portions of one or more exons of a Dystrophin gene, and the gene therapy vector (e.g., AAV gene therapy vectors) for the treatment of DMD comprises the one or more portions of the one or more exons of a Dystrophin gene that are not comprised in the genome of the subject suffering from DMD, to whom the gene therapy vector (e.g., AAV gene therapy vectors) for the treatment of DMD is administered.

In some aspects, the Dystrophin gene(s) comprised in the genome of the subject suffering from DMD, to whom the gene therapy vector (e.g., AAV gene therapy vectors) for the treatment of DMD is administered, lacks one or more exons of a Dystrophin gene, and the gene therapy vector (e.g., AAV gene therapy vectors) for the treatment of DMD comprises the one or more exons of a Dystrophin gene that are not comprised in the genome of the subject suffering from DMD, to whom the gene therapy vector (e.g., AAV gene therapy vectors) for the treatment of DMD is administered.

### Inverted Terminal Repeats

In some embodiments, the polynucleotides, plasmids, viral vectors, vector systems, viral packaging systems, cells, and compositions further comprise a nucleotide sequence comprising, consisting of, or consisting essentially of one or more inverted terminal repeats (ITRS). In some embodiments, the polynucleotides, plasmids, viral vectors, vector systems, viral packaging systems, cells, and compositions further comprise two, three, four, five, or six or more nucleotide sequences comprising, consisting of, or consisting essentially of two, three, four, five, or six or more ITRs. In some embodiments, the two or more ITRs are the same. In some embodiments, the two or more ITRs are different.

In some embodiments, the recombinant polynucleotide is flanked by the two or more ITRs. In some embodiments, the transgene of interest is flanked by a pair of ITRs. In some embodiments, the expression cassette sequence is flanked by the pair of ITRs. In some embodiments, the ITRs in the first pair of ITRs are the same. In some embodiments, the ITRs in the first pair of ITRs are different. In some embodiments, the ITRs in the second pair of ITRs are the same. In some embodiments, the ITRs in the second pair of ITRs are different. In some embodiments, the ITRs in the first pair of ITRs are the same as the ITRs in the second pair of ITRs. In some embodiments, at least one ITR in the first pair of ITRs is the same as at least one ITR in the second pair of ITRs. In some embodiments, the ITRs in the first pair of ITRs are different from the ITRs in the second pair of ITRs. In some embodiments, at least one ITR in the first pair of ITRs is different from at least one ITR in the second pair of ITRs.

In some embodiments, the ITR is a viral ITR. In some embodiments, the ITR is an AAV ITR. In some embodiments, the AAV ITR is selected from an ITR from at least one of AAV serotypes AAVrh.20, AAV-1, AAV-2, AAV-3, AAV-4, AAV-5, AAV-6, AAV-7, AAVrh.74, AAV-8, AAV-9, AAV-10, AAVrh. 10, AAV-11, AAV-12 and AAV-13. In some embodiments, the AAV ITR is an AAV2 ITR. In some embodiments, the AAV ITR is an AAV5 ITR. The ITR sequences for AAV1-6 can be found, for example, in Grimm et al., J. Virol. 80 (1): 426-39 (2006), which is incorporated by reference in its entirety.

In some embodiments, the recombinant polynucleotide does not comprise an AAV sequence other than an inverted terminal repeat (ITR).

An example sequence of a 5′ ITR from an AAV2 is:

(SEQ ID NO: 14)
GCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGG
GAGTGGCCAACTCCATCACTAGGGGTTCCTTGTAGTTAATGATTAAC.

A corresponding 3′ ITR from an AAV2 is:

(SEQ ID NO: 15)
GTTAATCATTAACTACAAGGAACCCCTAGTGATGGAGTTGGCCACTCCC
TCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGC.

### Promoters

In some embodiments, the polynucleotides, plasmids, viral vectors, vector systems, viral packaging systems, cells, and compositions further comprise a nucleotide sequence comprising, consisting of, or consisting essentially of one or more promoters. In some embodiments, the promoter is a eukaryotic promoter. Examples of eukaryotic promoters include, but are not limited to, a cytomegalovirus (CMV) promoter, elongation factor 1 alpha (EF1a) promoter, CAG promoter, phosphoglycerate kinase gene (PGK) promoter, tetracycline response element (TRE) promoter, human U6 nuclear (U6) promoter, and UAS promoter. In some embodiments, the promoter is a mammalian promoter. In some embodiments, the promoter is a constitutive promoter. In some embodiments, the promoter is an inducible promoter.

In some embodiments, the promoter is a tissue-specific promoter. Examples of tissues include, but are not limited to, muscle, epithelial, connective, and nervous tissue. Examples of tissue-specific promoters include, but are not limited to, B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, ICAM-2 promoter, INF-β promoter, Mb promoter, NphsI promoter, OG-2 promoter, SP-B promoter, SYN1 promoter, WASP promoter, SV40/bAlb promoter, SV40/hAlb promoter, SV40/CD43 promoter, SV40/CD45 promoter, and NSE/RU5′ promoter.

In some embodiments, the promoter is a muscle-specific promoter. In some embodiments, the muscle-specific promoter is a myosin heavy chain complex—E box muscle creatine kinase fusion enhancer/promoter.

In some embodiments, the promoter is a recombinant promoter. In some embodiments, the recombinant promoter is a recombinant muscle-specific promoter. In some embodiments, the recombinant-muscle specific promoter is a recombinant myosin heavy chain-creatine kinase muscle-specific promoter. In another embodiment, the muscle-specific promoter comprises a human skeletal actin gene element, a cardiac actin gene element, a desmin promoter, a skeletal alpha-actin (ASKA) promoter, a troponin I (TNNI2) promoter, a myocytespecific enhancer binding factor mef binding element, a muscle creatine kinase (MCK) promoter, a truncated MCK (tMCK) promoter, a myosin heavy chain (MHC) promoter, a hybrid a-myosin heavy chain enhancer-/MCK enhancer-promoter (MHCK7) promoter, a C5-12 promoter, a murine creatine kinase enhancer element, a skeletal fast-twitch troponin c gene element, a slow-twitch cardiac troponin c gene element, a slow-twitch troponin i gene element, hypoxia-inducible nuclear factor.

An example of an MHCK7 promoter/enhancer is:

(SEQ ID NO: 12)
AAGCTTGCATGTCTAAGCTAGACCCTTCAGATTAAAAATAACTGAGGTA
AGGGCCTGGGTAGGGGAGGTGGTGTGAGACGCTCCTGTCTCTCCTCTAT
CTGCCCATCGGCCCTTTGGGGAGGAGGAATGTGCCCAAGGACTAAAAAA
AGGCCATGGAGCCAGAGGGGCGAGGGCAACAGACCTTTCATGGGCAAAC
CTTGGGGCCCTGCTGTCTAGCATGCCCCACTACGGGTCTAGGCTGCCCA
TGTAAGGAGGCAAGGCCTGGGGACACCCGAGATGCCTGGTTATAATTAA
CCCAGACATGTGGCTGCCCCCCCCCCCCCAACACCTGCTGCCTCTAAAA
ATAACCCTGTCCCTGGTGGATCCCCTGCATGCGAAGATCTTCGAACAAG
GCTGTGGGGGACTGAGGGCAGGCTGTAACAGGCTTGGGGGCCAGGGCTT
ATACGTGCCTGGGACTCCCAAAGTATTACTGTTCCATGTTCCCGGCGAA
GGGCCAGCTGTCCCCCGCCAGCTAGACTCAGCACTTAGTTTAGGAACCA
GTGAGCAAGTCAGCCCTTGGGGCAGCCCATACAAGGCCATGGGGCTGGG
CAAGCTGCACGCCTGGGTCCGGGGTGGGCACGGTGCCCGGGCAACGAGC
TGAAAGCTCATCTGCTCTCAGGGGCCCCTCCCTGGGGACAGCCCCTCCT
GGCTAGTCACACCCTGTAGGCTCCTCTATATAACCCAGGGGCACAGGGG
CTGCCCTCATTCTACCACCACCTCCACAGCACAGACAGACACTCAGGAG
CAGCCAGC.

### Introns

In some embodiments, the polynucleotides, plasmids, viral vectors, vector systems, viral packaging systems, cells, and compositions further comprise a nucleotide sequence comprising, consisting of, or consisting essentially of one or more introns. In some embodiments, the intron is a eukaryotic intron. In some embodiments, the intron is a mammalian intron. In some embodiments, the intron is a synthetic intron. In some embodiments, the intron is a chimeric intron. In some embodiments, the intron is from a non-coding exon. In some embodiments, the intron is upstream of or 5′ to the gene of interest.

In some embodiments, the intron comprises at least one of a 5′ donor site, branch point, or 3′ splice site. In some embodiments, the intron comprises two or more of a 5′ donor site, branch point, or 3′ splice site. In some embodiments, the intron comprises a 5′ donor site, branch point, and 3′ splice site.

In some embodiments, the intron comprises a 5′ donor site from a human β-globin gene.

In some embodiments, the intron comprises a branch point from an immunoglobulin G (IgG) heavy chain.

In some embodiments, the intron comprises a 3′ splice acceptor site from an immunoglobulin G (IgG) heavy chain.

In some embodiments, the intron is an SV40 intron. An example sequence of an SV40 intron is:

(SEQ ID NO: 13)
AGGTAAGTTTAGTCTTTTTGTCTTTTATTTCAGGTCCCGGATCCGGTGG
TGGTGCAAATCAAAGAACTGCTCCTCAGTGGATGTTGCCTTTACTTCTA
GGCCTGTACGGAAGTGTTACTTCTGCTCTAAAAGCTGCGGAATTGTACC
CGC.

### Selection Marker

In some embodiments, the polynucleotides, plasmids, viral vectors, vector systems, viral packaging systems, cells, and compositions further comprise a nucleotide sequence comprising, consisting of, or consisting essentially of one or more selection markers. In some embodiments, the selection marker is a bacterial selectable marker. In some embodiments, the selection marker is an antibiotic resistance gene. Examples of antibiotic resistance genes include, but are not limited to, β-lactamase, kanamycin resistance gene, neo gene from Tn5, mutant FabI gene from E. coli genome, and URA3 (an orotidine-5′ phosphate decarboxylase from yeast). In some embodiments, the antibiotic resistance gene is a β-lactamase gene. In some embodiments, the antibiotic resistance gene is a kanamycin resistance gene.

### Polyadenylation Signal

In some embodiments, the polynucleotides, plasmids, viral vectors, vector systems, viral packaging systems, cells, and compositions further comprise a nucleotide sequence comprising, consisting of, or consisting essentially of one or more polyadenylation (polyA) signals. In some embodiments, the polyA signal is an artificial polyA signal. An example polyA signal sequence is:

(SEQ ID NO: 16)
GGCCGCAATAAAAGATCTTTATTTTCATTAGATCTGTGTGTTGGTTTTT
TGTG.

### Packaging Systems

In some embodiments, the adeno-associated viral packaging system comprises: (a) a plasmid encoding an AAV expression cassette; (b) a plasmid encoding AAV capsid and Rep genes, and (c) an adenovirus helper plasmid. In some embodiments, the adenovirus helper plasmid comprises one or more genes from an adenovirus. In some embodiments, the one or more genes from the adenovirus mediate AAV replication. In some embodiments, the one or more genes from the adenovirus are selected from E4, E2a, and VA.

### Compositions

Further disclosed herein are compositions comprising, consisting of, or consisting essentially of any of the polynucleotides disclosed herein. Further disclosed herein are compositions comprising, consisting of, or consisting essentially of any of the AAV vectors disclosed herein.

Further disclosed herein is a composition comprising, consisting of, or consisting essentially of: (a) a recombinant adeno-associated virus (rAAV) vector, wherein the rAAV vector comprises, consists of, or consists essentially of any of the polynucleotides disclosed herein; and (b) a pharmaceutically acceptable carrier, diluent, excipient, or adjuvant.

In some embodiments, any of the compositions disclosed herein further comprise at least one of a pharmaceutically acceptable carrier, diluent, excipient, or adjuvant. Acceptable carriers, diluents and adjuvants are nontoxic to recipients and are preferably inert at the dosages and concentrations employed and include buffers and surfactants such as pluronics. Examples of acceptable carriers include, but are not limited to, phosphate buffered saline, preservatives, and the like.

The pharmaceutically acceptable carrier, diluent, or excipient may be suitable for injectable use. Examples of pharmaceutically acceptable carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, poloxamer and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the polynucleotides, plasmids, viral vectors, or dual vector systems disclosed herein in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

### Methods of Producing AAV Vectors

Disclosed herein are methods of producing an adeno-associated viral (AAV) vector (e.g., virus or viral particle). Methods of producing AAV vectors are known in the art. For instance, such methods are disclosed in, for example, WO 01/83692, which is incorporated by reference herein in its entirety. General principles of AAV production are reviewed in, for example, Carter, Current Opinion in Biotechnology 3:533-539 (1992); and Muzyczka, N., Curr. Topics in Microbiol. and Immunol. 158:97-129 (1992), each of which are incorporated by reference in their entirety. Various approaches for producing AAVs are described in Ratschin et al., Mol. Cell. Biol. 4:2072 (1984); Hermonat et al., Proc. Natl. Acad. Sci. USA 81:6466 (1984); Tratschin et al., Mol. Cell. Biol. 5:3251 (1985); Mclaughlin et al., J. Virol., 62:1963 (1988); and Lebkowski et al., Mol. Cell. Biol. 7:349 (1988); Samulski et al., J. Virol. 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-1250 (1995); Paul et al., Human Gene Therapy 4:609-615 (1993); Clark et al., Gene Therapy 3:1124-1132 (1996); U.S. Pat. Nos. 5,786,211; 5,871,982; and 6,258,595, each of which are incorporated by reference in their entirety.

In some embodiments, the method for producing an adeno-associated viral (AAV) vector comprises transducing a cell with any of the AAV packaging systems disclosed herein. In some embodiments, the cell is a eukaryotic cell. In some embodiments, the cell is a mammalian cell and in other embodiments the cell is an insect cell. In some embodiments, the cell is a recombinant cell that stably expresses the adeno-associated virus rep and cap genes. In some embodiments, the method further comprises culturing the cell to produce a population of transduced cells. In some embodiments, the method further comprises collecting the supernatant from the population of transduced cells. In some embodiments, the method further comprises subjecting the supernatant to one or more purification steps to produce a purified AAV vector sample, wherein the AAV vector sample is substantially free from cellular debris and proteins. Alternatively, or additionally, the method further comprises lysing the population of transduced cells to produce a cellular lysate. In some embodiments, the method further comprises subjecting the cellular lysate to one or more purification steps to produce a purified AAV vector sample, wherein the AAV vector sample is substantially free from cellular debris and proteins. In some embodiments, the purity of the purified AAV vector sample is at least 85%, 86%, 87%, 88%, 89%, 90%, 91%, 92%, 93%, 94%, 95%, 96%, 97%, 98%, 99% or 100% pure.

### Cells

Further disclosed herein are cells comprising any of the polynucleotides disclosed herein. The cells can be prokaryotic or eukaryotic cells. Non-limiting examples of eukaryotic cells include mammalian, e.g., hamster, murine, rat, canine, ovine or human cells. In some embodiments, the cells are transfected with a plasmid comprising any of the polynucleotides disclosed herein. In some embodiments, the cells are transduced with any of the AAV expression cassettes disclosed herein. In some embodiments, the cells are infected with any of the AAV vectors disclosed herein.

Any of the cells disclosed herein may be packaging cells that produce infectious rAAV. In some embodiments, the packaging cells are stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells). Non-limiting examples of prokaryotic cells comprise bacterial cells (e.g., Escherichia coli) and archaeal cells. The cells of the disclosure can be used to produce a cell bank, e.g., an Accession Cell Banks (ACB) for non-GMP purpose or GMP Master Cell Bank (MCB). The aliquote of the cells, in one embodiment, are expanded from an original inoculum to a larger volume before culture in the bioreactor for the production.

### Methods of Treatment

Further disclosed herein are methods of treating a muscle disease or disorder which comprises, consists of, or consists essentially of administering to a subject in need thereof an effective amount of an adeno-associated viral (AAV) vector. In some embodiments, the AAV vector is administered orally, parenterally (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.). In some embodiments, the AAV vector is administered intramuscularly or intravenously.

Further disclosed herein are uses of any of the recombinant polynucleotides, plasmids, viral vectors, vector systems, and compositions in the manufacture of a medicament to treat a muscle disease or disorder in a subject in need thereof.

Titers of AAV vectors to be administered in methods of the invention will vary depending, for example, on the particular AAV, the mode of administration, the treatment goal, the individual, and the cell type(s) being targeted, and may be determined by methods standard in the art. Titers of AAV may range from at least about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). For instance, dosages of AAV may range from at least about 1×10⁶, about 1×10⁷, about 1×10⁸, about 1×10⁹, about 1×10¹⁰, about 1×10¹¹, about 1×10¹², about 2×10¹², about 3×10¹², about 4×10¹², about 5×10¹², about 6×10¹², about 7×10¹², about 8×10¹², about 9×10¹², about 1×10¹³ to about 1×10¹⁴ viral genomes.

AAV dosage can be determined by multiple methods, which include but are not limited to ELISA, assessment of the reverse transcriptase activity, FACS, transduction assays northern blotting (e.g., semi-quantitative northern), dot blot analysis or PCR (e.g., qPCR). It is well known that the AAV doses can be determined by measuring AAV vector genomes with quantitative real-time PCR (qPCR). Such qPCR methods overcome the inconsistency or arbitrary results from conventional transduction assays. In one embodiment of PCR dosage determination, plasmid DNA is used as a calibration standard. The forms of the plasmids can impact the dosage results from the qPCR methods. In one embodiment, the circular or supercoiled DNA or plasmids are used as a quantification standard.

In some embodiment, dosages may be expressed in the units of vg/kg, based on a supercoiled DNA or plasmid as the quantitation standard. For example, dosages of AAV is about 1×10⁶-1×10¹⁶ vg/kg, about 1×10⁸-1×10¹⁵ vg/kg, or about 1×10¹⁰-1×10¹⁴ vg/kg,), based on a supercoiled DNA or plasmid as the quantitation standard. In another embodiment, the dosages is about at least 1×10⁶, about 1×10⁷, about 1×10⁸, about 1×10⁹, about 1×10¹⁰, about 1×10¹¹, about 1×10¹², about 2×10¹², about 4×10¹², about 6×10¹², about 8×10¹², about 1×10¹³, about 2×10¹³, about 2.4×10¹³, about 3×10¹³, about 4×10¹³, about 5×10¹³, about 6×10¹³, about 7×10¹³, about 8×10¹³, about 9×10¹³, about 1×10¹⁴, about 1×10¹⁵, or at least about 1×10¹⁶ vg/kg. In one embodiment, the dosage is at least 2×10¹², 4×10¹², 6×10¹², 8×10¹², 1×10¹³, 2×10¹³, 2.4×10¹³, 3×10¹³, 4×10¹³, 5×10¹³, 6×10¹³, 7×10¹³, or 8×10¹³ vg/kg, based on a supercoiled DNA or plasmid as the quantitation standard.

In some embodiments, the methods disclosed herein comprise administering at least about 1×10⁶, about 1×10⁷, about 1×10⁸, about 1×10⁹, about 1×10¹⁰, about 1×10¹¹, about 1×10¹², about 2×10¹², about 3×10¹², about 4×10¹², about 5×10¹², about 6×10¹², about 7×10¹², about 8×10¹², about 9×10¹², about 1×10¹³ vg in a total volume of 1.5 ml per injection. In some embodiments, the methods disclosed herein comprise administering a total daily dose of at least about 1×10⁶, about 1×10⁷, about 1×10⁸, about 1×10⁹, about 1×10¹⁰, about 1×10¹¹, about 1×10¹², about 2×10¹², about 3×10¹², about 4×10¹², about 5×10¹², about 6×10¹², about 7×10¹², about 8×10¹², about 9×10¹², about 1×10¹³, about 2×10¹³, about 5×10¹³, about 7×10¹³, about 1×10¹⁴ vg. One exemplary method of determining encapsidated vector genome titer uses quantitative PCR, such as the methods described in Pozsgai et al., Mol. Ther. 25 (4): 855-869 (2017), which is incorporated by reference in its entirety.

In some embodiments, any of the polynucleotides, plasmids, viral vectors, dual vector systems, or compositions disclosed herein are administered to the subject at least 1, 2, 3, 4, 5, 6, 7, 8, 9, or 10 times a day. In some embodiments, any of the polynucleotides, plasmids, viral vectors, dual vector systems, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, plasmids, viral vectors, dual vector systems, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the polynucleotides, plasmids, viral vectors, dual vector systems, or compositions disclosed herein are administered to the subject at least every 1, 2, 3, 4, 5, 6, 7, 8, 9, or 10 days. In some embodiments, any of the polynucleotides, plasmids, viral vectors, dual vector systems, or compositions disclosed herein are administered to the subject at least every 1, 2, 3, 4, 5, 6, 7, 8, 9, 10, 11, 12, 13, or 14 weeks. In some embodiments, any of the polynucleotides, plasmids, viral vectors, dual vector systems, or compositions disclosed herein are administered to the subject for a period of at least 1, 2, 3, 4, 5, 6, 7, 8, 9, or 10 days. In some embodiments, any of the polynucleotides, plasmids, viral vectors, dual vector systems, or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the polynucleotides, plasmids, viral vectors, dual vector systems, or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the methods disclosed herein comprise administering any of the polynucleotides, plasmids, viral vectors, dual vector systems, or compositions disclosed herein systemically. For example, systemic administration is administration into the circulatory system so that the entire body is affected. Systemic administration includes enteral administration such as absorption through the gastrointestinal tract and parenteral administration through injection, infusion or implantation.

In some embodiments, the methods disclosed herein comprise administering any of the polynucleotides, plasmids, viral vectors, dual vector systems, or compositions disclosed herein locally. In some embodiments, the methods disclosed herein comprise administering any of the polynucleotides, plasmids, viral vectors, dual vector systems, or compositions disclosed herein to one or more tissues. In some embodiments, the tissue is selected from muscle, epithelial, connective, and nervous tissue. In some embodiments, the tissue is a muscle tissue.

In some embodiments, the methods disclosed herein comprise administering any of the polynucleotides, plasmids, viral vectors, dual vector systems, or compositions disclosed herein to the subject's foot. In some embodiments, the methods disclosed herein comprise administering any of the polynucleotides, plasmids, viral vectors, dual vector systems, or compositions disclosed herein to the subject's extensor digitorum brevis (EDB) muscle.

Combination therapies are also contemplated by the invention. Combination as used herein includes both simultaneous treatment and sequential treatments. Combinations of methods of the invention with standard medical treatments (e.g., corticosteroids) are specifically contemplated, as are combinations with novel therapies.

In some embodiments, the methods disclosed herein further comprise detecting the presence or absence of a mutation in a dystrophin gene in the subject prior to or subsequent to administering any of the polynucleotides, plasmids, viral vectors, dual vector systems, or compositions disclosed herein to the subject. In some embodiments, any of the polynucleotides, plasmids, viral vectors, dual vector systems, or compositions disclosed herein are administered to the subject upon detection of the presence of the mutation in the dystrophin gene.

In some embodiments, the methods disclosed herein further comprise detecting levels of dystrophin protein in the subject prior to administering or subsequent to any of the polynucleotides, plasmids, viral vectors, dual vector systems, or compositions disclosed herein to the subject. In some embodiments, the methods disclosed herein further comprise detecting levels of the transgene protein in the subject after administering any of the polynucleotides, plasmids, viral vectors, dual vector systems, or compositions disclosed herein to the subject. In some embodiments, detecting the levels of the transgene comprises detecting expression of the dysferlin gene. Detecting expression of the transgene may comprise quantifying the transgene DNA or RNA levels. Alternatively, or additionally, detecting the levels of the transgene protein comprises quantifying the levels of the transgene protein. In some embodiments, the levels of the transgene protein are detected in a sample from the subject. In some embodiments, the sample is a body fluid sample. Examples of body fluid samples include, but are not limited to, blood, urine, sweat, saliva, stool, and synovial fluid. In some embodiments, the blood sample is a plasma or serum sample. In some instances, the method further comprises a DNA sequencing test, e.g., from Athena Diagnostics (CPT: 81408 (1)).

In some embodiments, the methods disclosed herein further comprise modifying the dose or dosing frequency of any of the polynucleotides, plasmids, viral vectors, dual vector systems, or compositions that is administered to the subject.

### Kits

In a yet further aspect, a kit is provided that comprises, or alternatively consists essentially of, or yet further consisting of, any of one or more of the polynucleotides, polypeptides, vectors, cells and systems, or the compositions, and instructions for use. In one aspect, any of one or more of the polynucleotides, polypeptides, vectors, cells and systems, or the compositions are detectably labeled or further comprise a purification or detectable marker. In some instances, the kit comprises a) a polynucleotide, wherein the polynucleotide is the recombinant polynucleotide described herein; or b) a adeno-associated viral (AAV) vector, wherein the AAV vector is the AAV vector described herein; or c) a composition described herein; or d) a cell (e.g., a host cell, optionally mammalian cell) described herein; and optionally an instruction for use.

### EXAMPLES

### Example 1: Evaluating the Safety of Myotropic Capsids

An AAV9 capsid with the insertion of the peptide ENRRGDFNNT (“4E,” SEQ ID NO: 2) between positions 585 and 589 (i.e., positions 586-588 of the AAV9 capsid protein are replaced or substituted by the 4E peptide) has shown promising efficacy for transducing muscle tissues in pre-clinical experiments. However, some attempts to develop AAV9 capsids for clinical applications have not been successful, mostly due to safety concerns.

This myotropic AAV9 capsid, “MyoAAV-4E,” was evaluated for safety and efficacy as compared to a wild type rh74 capsid. Both rAAVs carried the same expression cassette of a primate microdystrophin gene under the control of an MHCK7 promoter. This primate microdystrophin has a sequence of:

(SEQ ID NO: 9)
MLWWEEVEDCYEREDVQKKTFTKWVNAQFSKFGKQHIENLFSDLQDGRR
LLDLLEGLTGQKLPKEKGSTRVHALNNVNKALRVLQNNNVDLVNIGSTD
IVDGNHKLTLGLIWNIILHWQVKNVMKNIMAGLQQTNSEKILLSWVRQS
TRNYPQVNVINFTTSWSDGLALNALIHSHRPDLFDWNSVVCQQSATQRL
EHAFNIARYQLGIEKLLDPEDVATTYPDKKSILMYITSLFQVLPQQVSI
EAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDR
YQTALEEVLSWLLSAEDTLQAQGEISNDVEVVKEQFHTHEGYMMDLTAH
QGRVGNILQLGSQLIGTGKLSEDEETEVQEQMNLLNSRWECLRVASMEK
QSNLHRVLMDLQNQKLKELNDWLTKTEERTRKMEKEPLGPDLEDLKRQV
QQHKVLQEDLEQEQVRVNSLTHMVVVVDESSGDHATAALEEQLKVLGDR
WANICRWTEDRWVLLQDILLKWQRLTEEQCLFSAWLSEKEDVVNRIHTT
GFKDQNEMLSSLQKLAVLKADLEKKKQSMDKLYSLKQDLLSTLKNKSVT
QKMEAWLENFARCWDNLVQKLEKSTAQISQAVTTTQPSLTQTTVMETVT
MVTTREQILVKHAQEELPPPPPQKKRTLERLQELQEATDELDLKLRQAE
VIKGSWQPVGDLLIDSLQDHLEKVKALRGEIAPLKENVSHVNDLARQLT
TLGIQLSPYNLSTLEDLNTRWKLLQVAVEDRVRQLHEAHRDFGPASQHE
LSTSVQGPWERAISPNKVPYYINHETQTTCWDHPKMTELYQSLADLNNV
RFSAYRTAMKLRRLQKALCLDLLSLSAACDALDQHNLKQNDQPMDILQI
INCLTTIYDRLEQEHNNLVNVPLCVDMCLNWLLNVYDTGRTGRIRVLSF
KTGIISLCKAHLEDKYRYLFKQVSSSTGFCDQRRLGLLLHDSIQIPRQL
GEVASFGGSNIEPSVRSCFQFANNKPEIEAALFLDWMRLEPQSMVWLPV
LHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYL
PVQTVLEGDNMETDTM.

An exemplary nucleotide sequence encoding SEQ ID NO: 9 is:

(SEQ ID NO: 10)
ATGCTCTGGTGGGAAGAAGTTGAAGACTGCTACGAGCGCGAAGATGTACAAAAGAAGACC
TTCACCAAATGGGTCAATGCACAGTTTTCCAAATTTGGTAAACAGCACATCGAGAACCTT
TTCTCTGATCTGCAGGATGGCAGACGGCTGCTGGACCTGCTAGAGGGGCTGACTGGTCAG
AAACTCCCCAAGGAGAAAGGTTCTACACGCGTCCATGCGCTTAACAATGTGAATAAGGCT
CTCCGAGTACTTCAGAACAATAATGTAGATTTGGTGAATATAGGATCTACTGATATTGTT
GACGGAAACCACAAACTGACATTAGGTCTGATCTGGAATATTATCCTGCACTGGCAGGTG
AAAAATGTGATGAAGAACATCATGGCCGGCCTCCAACAGACCAATTCAGAAAAGATATTA
CTTAGCTGGGTGAGGCAGAGCACTAGGAACTACCCCCAGGTTAATGTAATTAATTTCACA
ACGAGCTGGAGCGATGGGCTCGCACTGAATGCGTTGATCCACTCCCACAGACCTGATTTG
TTTGACTGGAACTCGGTTGTCTGTCAGCAATCCGCCACTCAGCGGCTGGAGCATGCCTTC
AACATTGCAAGATATCAGCTCGGGATTGAAAAATTACTCGATCCAGAGGACGTGGCCACC
ACCTACCCAGACAAGAAGAGCATCCTCATGTACATCACTAGCCTCTTCCAAGTTTTGCCT
CAACAGGTCAGCATTGAGGCCATTCAGGAGGTGGAGATGCTGCCCAGGCCTCCAAAGGTC
ACTAAAGAGGAGCATTTTCAACTCCACCACCAGATGCATTATAGTCAGCAGATAACCGTG
TCGTTAGCTCAGGGTTATGAGAGGACCAGTAGCCCTAAGCCCCGCTTCAAGTCCTACGCT
TACACCCAGGCTGCCTATGTCACAACTAGCGACCCAACTAGGTCACCGTTCCCGTCACAA
CACCTTGAAGCCCCCGAGGATAAATCTTTTGGAAGTTCCCTGATGGAATCAGAAGTGAAC
TTGGACAGGTACCAGACTGCTTTAGAAGAGGTCCTGTCCTGGCTGCTGAGTGCCGAGGAC
ACCCTGCAAGCACAAGGTGAAATATCGAATGACGTCGAGGTGGTTAAAGAACAGTTCCAC
ACTCATGAGGGCTACATGATGGATCTCACAGCACACCAGGGCCGGGTGGGAAACATTCTG
CAGCTGGGAAGTCAGCTCATAGGAACAGGCAAGCTCAGTGAAGACGAGGAAACAGAAGTA
CAGGAGCAGATGAACCTGCTTAACAGCAGGTGGGAGTGTCTGCGGGTTGCATCAATGGAG
AAACAGAGTAACCTGCATCGAGTCTTGATGGATTTACAAAACCAGAAACTGAAAGAACTG
AACGATTGGCTGACGAAGACGGAGGAGCGTACTCGCAAGATGGAGAAGGAGCCGCTTGGA
CCAGACCTCGAAGACCTGAAGCGACAGGTGCAGCAGCATAAAGTGCTGCAGGAAGATCTT
GAGCAGGAACAAGTGCGGGTAAATTCCCTGACACACATGGTCGTAGTGGTGGATGAGAGC
AGTGGAGACCACGCCACAGCTGCTCTGGAGGAACAGTTAAAAGTGCTGGGCGACCGCTGG
GCCAATATTTGTAGATGGACTGAGGACCGATGGGTTCTATTGCAGGACATATTGCTAAAG
TGGCAGCGTCTAACCGAGGAACAGTGCCTGTTTTCTGCCTGGCTGTCTGAAAAGGAAGAT
GTGGTGAACAGAATTCATACCACTGGATTTAAAGATCAGAATGAAATGCTGTCCTCGCTA
CAGAAGCTGGCTGTGCTGAAAGCTGACCTAGAGAAGAAAAAACAGTCAATGGACAAACTC
TACTCCCTAAAACAAGACTTGCTGAGTACACTGAAGAACAAGTCTGTTACCCAGAAAATG
GAGGCCTGGTTAGAAAATTTTGCCCGCTGCTGGGACAACCTAGTCCAGAAGCTGGAAAAG
TCCACAGCCCAGATCTCCCAGGCCGTGACCACTACTCAGCCATCACTTACTCAGACAACC
GTCATGGAAACTGTCACCATGGTAACAACGCGGGAGCAGATACTGGTCAAGCATGCTCAA
GAAGAACTGCCTCCCCCGCCTCCCCAAAAGAAAAGGACTTTGGAGCGGTTGCAAGAACTC
CAAGAGGCAACCGATGAGCTTGACCTAAAACTCCGACAAGCTGAAGTGATTAAAGGGTCT
TGGCAGCCCGTGGGGGACCTCCTGATAGATTCTTTGCAAGATCATTTAGAAAAAGTCAAA
GCCCTGAGAGGTGAGATTGCTCCATTGAAGGAAAACGTGTCTCATGTTAATGACTTAGCG
AGACAGCTCACGACCCTTGGGATCCAGTTGTCCCCCTATAATCTTTCGACATTGGAGGAC
CTCAACACGCGTTGGAAGCTTCTGCAAGTTGCAGTAGAAGATAGAGTCCGTCAGCTGCAT
GAGGCGCACAGGGACTTTGGCCCAGCGTCTCAGCACTTTTTGAGCACATCCGTTCAGGGG
CCCTGGGAGCGCGCCATCTCACCCAACAAAGTTCCATACTATATTAACCATGAGACTCAA
ACCACCTGCTGGGATCACCCTAAAATGACCGAGCTCTATCAATCTCTGGCAGATTTGAAT
AATGTGCGGTTCTCAGCATATCGAACAGCCATGAAATTACGCCGCCTCCAAAAGGCGCTT
TGCTTGGATCTGCTCTCTCTCAGCGCAGCCTGTGATGCCCTCGACCAGCATAATTTGAAG
CAGAACGATCAGCCTATGGATATCTTACAAATCATCAACTGCCTAACGACCATCTATGAT
AGACTTGAGCAGGAACACAACAACCTGGTGAATGTGCCCCTCTGTGTGGACATGTGTCTG
AACTGGCTCCTCAATGTCTATGACACAGGGAGGACCGGCCGCATCCGAGTTCTGAGTTTT
AAGACAGGAATTATCTCCCTTTGTAAAGCCCACCTGGAGGATAAGTACAGGTACCTGTTC
AAGCAAGTTTCATCTTCCACCGGCTTTTGTGACCAGAGAAGACTCGGTTTACTGCTTCAT
GATAGTATACAAATCCCTAGACAGTTGGGAGAGGTGGCCTCTTTTGGGGGCAGCAACATT
GAGCCCTCTGTCAGAAGCTGCTTCCAGTTTGCCAATAATAAACCTGAAATTGAAGCAGCT
TTGTTCCTGGACTGGATGAGCTTGGAGCCACAGAGCATGGTGTGGCTGCCTGTCCTGCAC
CGAGTAGCCGCTGCAGAGACAGCAAAGCATCAGGCGAAATGCAATATCTGCAAGGAGTGT
CCGATCATTGGGTTCCGGTACCGGTCACTGAAGCACTTCAATTATGATATCTGCCAGAGT
TGTTTCTTCAGCGGGCGGGTAGCCAAGGGCCACAAGATGCACTATCCTATGGTCGAGTAC
TGCACCCCAACTACCAGCGGCGAGGACGTGAGAGATTTTGCTAAAGTGCTAAAGAACAAA
TTTAGAACAAAACGCTACTTCGCTAAGCATCCAAGGATGGGCTATCTTCCTGTGCAAACA
GTGTTGGAAGGAGACAACATGGAAACAGACACCATGTGA.

The primate microdystrophin serves as a surrogate for human microdystrophin in experiments involving non-human primates and cells of a non-human origin. The human microdystrophin is:

(SEQ ID NO: 7)
MLWWEEVEDCYEREDVQKKTFTKWVNAQFSKFGKQHIENLFSDLQDGRR
LLDLLEGLTGQKLPKEKGSTRVHALNNVNKALRVLQNNNVDLVNIGSTD
IVDGNHKLTLGLIWNIILHWQVKNVMKNIMAGLQQTNSEKILLSWVRQS
TRNYPQVNVINFTTSWSDGLALNALIHSHRPDLFDWNSVVCQQSATQRL
EHAFNIARYQLGIEKLLDPEDVDTTYPDKKSILMYITSLFQVLPQQVSI
EAIQEVEMLPRPPKVTKEEHFQLHHQMHYSQQITVSLAQGYERTSSPKP
RFKSYAYTQAAYVTTSDPTRSPFPSQHLEAPEDKSFGSSLMESEVNLDR
YQTALEEVLSWLLSAEDTLQAQGEISNDVEVVKDQFHTHEGYMMDLTAH
QGRVGNILQLGSKLIGTGKLSEDEETEVQEQMNLLNSRWECLRVASMEK
QSNLHRVLMDLQNQKLKELNDWLTKTEERTRKMEEEPLGPDLEDLKRQV
QQHKVLQEDLEQEQVRVNSLTHMVVVVDESSGDHATAALEEQLKVLGDR
WANICRWTEDRWVLLQDILLKWQRLTEEQCLFSAWLSEKEDAVNKIHTT
GFKDQNEMLSSLQKLAVLKADLEKKKQSMGKLYSLKQDLLSTLKNKSVT
QKTEAWLDNFARCWDNLVQKLEKSTAQISQAVTTTQPSLTQTTVMETVT
TVTTREQILVKHAQEELPPPPPQKKRTLERLQELQEATDELDLKLRQAE
VIKGSWQPVGDLLIDSLQDHLEKVKALRGEIAPLKENVSHVNDLARQLT
TLGIQLSPYNLSTLEDLNTRWKLLQVAVEDRVRQLHEAHRDFGPASQHE
LSTSVQGPWERAISPNKVPYYINHETQTTCWDHPKMTELYQSLADLNNV
RFSAYRTAMKLRRLQKALCLDLLSLSAACDALDQHNLKQNDQPMDILQI
INCLTTIYDRLEQEHNNLVNVPLCVDMCLNWLLNVYDTGRTGRIRVLSF
KTGIISLCKAHLEDKYRYLFKQVASSTGFCDQRRLGLLLHDSIQIPRQL
GEVASFGGSNIEPSVRSCFQFANNKPEIEAALFLDWMRLEPQSMVWLPV
LHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYL
PVQTVLEGDNMETDTM.

An exemplary nucleotide sequence encoding SEQ ID NO: 7 is:

(SEQ ID NO: 8)
ATGCTGTGGTGGGAGGAGGTGGAGGATTGTTATGAAAGGGAGGACGTGCAGAAGAAGACT
TTTACCAAGTGGGTGAACGCTCAGTTCAGCAAATTTGGGAAGCAGCACATCGAGAATCTG
TTTTCCGACCTGCAGGATGGGAGACGGCTGCTGGATCTGCTGGAAGGACTGACTGGCCAG
AAGCTGCCCAAAGAGAAGGGGAGCACTAGGGTGCACGCCCTGAACAACGTGAACAAAGCT
CTGAGAGTGCTGCAGAACAACAACGTGGATCTGGTGAATATTGGCAGTACTGATATCGTG
GACGGGAACCACAAACTGACACTGGGCCTGATCTGGAACATTATTCTGCACTGGCAGGTG
AAAAATGTGATGAAGAACATCATGGCCGGGCTGCAGCAGACCAATTCCGAGAAGATCCTG
CTGTCTTGGGTGCGGCAGAGCACCCGCAACTATCCCCAGGTGAACGTGATTAACTTCACT
ACATCCTGGAGCGACGGGCTGGCCCTGAATGCTCTGATTCACAGCCACAGGCCTGATCTG
TTCGACTGGAATAGCGTGGTGTGCCAGCAGTCTGCCACACAGCGCCTGGAACATGCCTTC
AATATCGCTCGGTACCAGCTGGGGATCGAAAAACTGCTGGACCCAGAGGATGTGGACACT
ACATACCCAGATAAAAAGTCTATTCTGATGTACATTACTAGCCTGTTCCAGGTGCTGCCA
CAGCAGGTGTCTATTGAAGCCATTCAGGAGGTGGAAATGCTGCCCCGCCCCCCCAAAGTG
ACTAAAGAGGAGCATTTTCAGCTGCATCATCAGATGCATTACAGCCAGCAGATTACCGTG
AGCCTGGCTCAGGGATATGAGCGCACCAGTAGTCCAAAACCACGGTTCAAGTCCTACGCT
TATACCCAGGCTGCCTACGTGACAACTAGCGACCCTACTAGATCCCCCTTTCCATCCCAG
CACCTGGAGGCCCCAGAGGACAAGAGCTTTGGGTCCAGCCTGATGGAAAGCGAGGTGAAT
CTGGATCGGTACCAGACAGCCCTGGAGGAGGTGCTGAGCTGGCTGCTGAGTGCTGAAGAC
ACACTGCAGGCCCAGGGCGAAATTTCCAATGACGTGGAAGTGGTGAAGGATCAGTTCCAC
ACACACGAGGGCTATATGATGGACCTGACAGCTCACCAGGGGCGCGTGGGCAATATCCTG
CAGCTGGGCTCTAAACTGATCGGCACCGGGAAACTGAGTGAGGACGAGGAAACAGAAGTG
CAGGAGCAGATGAACCTGCTGAACAGCCGCTGGGAGTGTCTGAGAGTGGCTAGTATGGAG
AAGCAGTCCAACCTGCACCGGGTGCTGATGGACCTGCAGAACCAGAAACTGAAAGAGCTG
AACGACTGGCTGACAAAGACTGAGGAACGCACAAGGAAGATGGAGGAGGAGCCACTGGGA
CCCGACCTGGAGGATCTGAAGAGACAGGTGCAGCAGCATAAGGTGCTGCAGGAGGATCTG
GAACAGGAGCAGGTGCGGGTGAACTCCCTGACACATATGGTGGTGGTGGTGGACGAATCT
AGTGGAGATCACGCCACCGCCGCCCTGGAGGAACAGCTGAAGGTGCTGGGGGACCGGTGG
GCCAACATTTGCCGGTGGACCGAGGACAGGTGGGTGCTGCTGCAGGACATCCTGCTGAAA
TGGCAGAGGCTGACCGAGGAGCAGTGTCTGTTTAGTGCTTGGCTGAGCGAGAAAGAGGAC
GCCGTGAACAAGATCCACACAACCGGCTTTAAGGATCAGAACGAAATGCTGTCTAGCCTG
CAGAAACTGGCTGTGCTGAAGGCCGATCTGGAGAAAAAGAAGCAGAGCATGGGCAAACTG
TATAGCCTGAAACAGGACCTGCTGAGCACCCTGAAGAACAAGAGCGTGACCCAGAAGACA
GAAGCCTGGCTGGATAACTTTGCCCGCTGCTGGGACAACCTGGTGCAGAAACTGGAGAAA
AGTACAGCTCAGATCTCTCAGGCTGTGACCACAACCCAGCCTAGCCTGACCCAGACAACC
GTGATGGAAACCGTGACCACCGTGACAACCCGCGAACAGATCCTGGTGAAACATGCCCAG
GAAGAGCTGCCACCTCCACCTCCCCAGAAGAAGAGAACCCTGGAGCGGCTGCAGGAGCTG
CAGGAAGCCACTGACGAACTGGACCTGAAGCTGAGGCAGGCCGAAGTGATTAAGGGGTCT
TGGCAGCCTGTGGGCGATCTGCTGATTGATTCCCTGCAGGACCACCTGGAAAAGGTGAAG
GCTCTGAGAGGCGAAATTGCTCCACTGAAGGAGAACGTGAGTCATGTGAACGATCTGGCT
AGACAGCTGACAACACTGGGCATCCAGCTGAGCCCATACAATCTGAGCACACTGGAGGAC
CTGAATACCAGGTGGAAGCTGCTGCAGGTGGCTGTGGAAGACCGGGTGCGGCAGCTGCAT
GAGGCCCATCGCGACTTCGGACCAGCCAGCCAGCACTTTCTGAGCACATCCGTGCAGGGG
CCCTGGGAGAGGGCCATTTCTCCCAACAAGGTGCCCTACTATATTAATCACGAGACCCAG
ACCACTTGTTGGGACCATCCCAAGATGACAGAACTGTACCAGTCCCTGGCCGATCTGAAC
AACGTGAGGTTTAGCGCTTACAGAACCGCTATGAAGCTGAGACGGCTGCAGAAGGCCCTG
TGCCTGGATCTGCTGTCCCTGTCCGCCGCCTGCGATGCCCTGGATCAGCATAATCTGAAG
CAGAACGATCAGCCAATGGATATCCTGCAGATCATCAACTGCCTGACCACTATCTACGAC
AGGCTGGAGCAGGAGCACAACAACCTGGTGAACGTGCCTCTGTGCGTGGATATGTGCCTG
AACTGGCTGCTGAACGTGTATGACACTGGGCGCACCGGCCGGATCAGAGTGCTGAGTTTT
AAAACTGGGATTATCTCCCTGTGTAAGGCCCACCTGGAGGACAAGTACAGGTACCTGTTC
AAGCAGGTGGCTAGTAGCACTGGATTTTGTGACCAGCGCCGCCTGGGACTGCTGCTGCAT
GATAGTATCCAGATTCCTAGACAGCTGGGAGAGGTGGCTAGTTTCGGAGGATCTAACATC
GAACCCAGCGTGCGCAGCTGTTTCCAGTTTGCCAATAACAAACCTGAAATCGAGGCTGCT
CTGTTCCTGGATTGGATGCGCCTGGAACCACAGAGCATGGTGTGGCTGCCTGTGCTGCAC
AGAGTGGCTGCCGCCGAAACTGCCAAGCACCAGGCTAAATGCAACATCTGCAAGGAATGT
CCCATTATCGGCTTTCGCTACAGGAGTCTGAAACATTTTAACTACGATATTTGCCAGAGC
TGCTTCTTTTCCGGAAGAGTGGCCAAAGGACACAAGATGCACTACCCTATGGTGGAATAT
TGCACCCCAACTACATCTGGCGAAGATGTGCGCGATTTTGCCAAGGTGCTGAAGAATAAG
TTTCGGACTAAGAGGTACTTCGCCAAGCACCCCCGCATGGGGTATCTGCCAGTGCAGACA
GTGCTGGAAGGAGACAATATGGAGACCGATACAATGTGA.

Cynomolgus macaques were injected with the capsids at the following doses: AAV rh74 at 2×10¹⁴ vector genomes per kilogram body weight (vg/kg), MyoAAV-4E at a low dose of 5×10¹³ vg/kg and a high dose of 2×10¹⁴ vg/kg. Saline was used as a negative control. Efficacy determinations were biodistribution as measured by vector copy number per nucleus and transduction efficiency measured by mRNA and protein quantification. Safety was determined by analysis of histopathology, complement activation, serum chemistry, hematology, ELISpot, electrocardiogram and nerve conduction velocity.

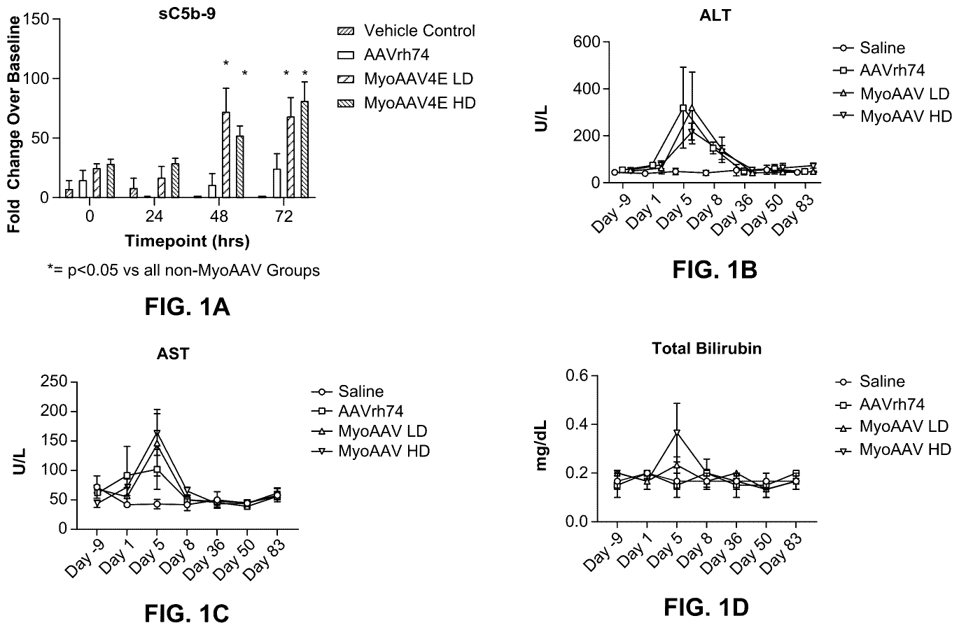

FIG. 1A demonstrates MyoAAV-4E injected animals had 50-100× complement change versus baseline at both the high and low doses, while the rh74 capsid did not produce any significantly elevated complement activity. Liver enzyme elevation was similar among all treatment groups (FIGS. 1B-1D).

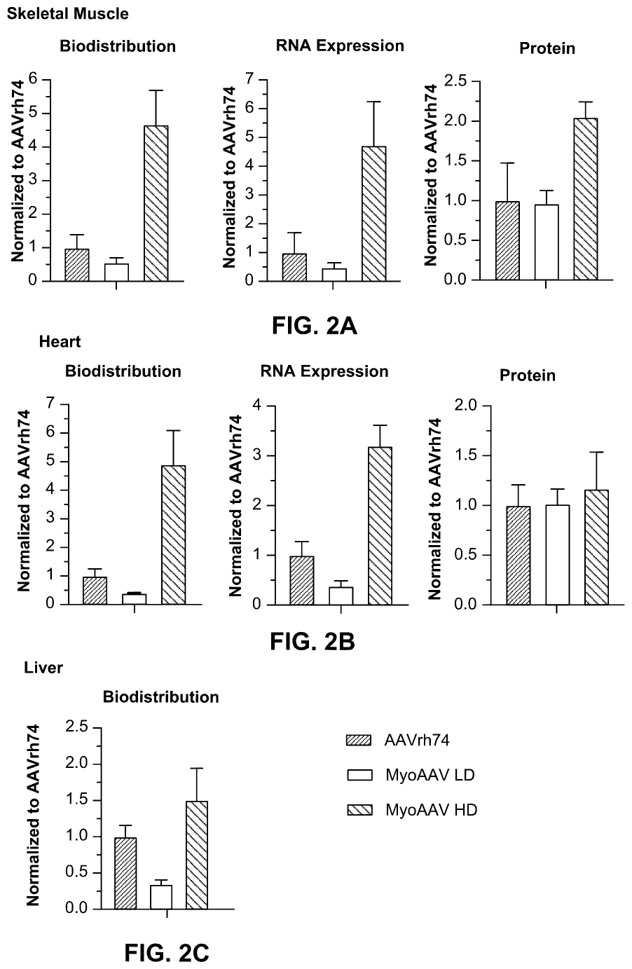

FIG. 2A provides enhanced biodistribution, RNA expression and protein levels in skeletal muscle with high dose MyoAAV-4E as compared to an equivalent dose of rh74. However, in the heart protein levels were the same between rh74 and both doses of MyoAAV-4E FIG. 2B). FIG. 2C shows biodistribution to the liver.

### Example 2: Generation of rh74 Myo Variants

The MyoAAV-4E capsid is produced poorly by transfected producer cells and the capsid displays unusual levels of aggregation under certain conditions. These characteristics make the MyoAAV-4E less suitable for clinical development.

The myotropic peptide ENRRGDFNNT (SEQ ID NO: 2) is inserted into an AAV rh74 capsid sequence in four different capsid variants to assess if an rh74-based capsid would produce better, be more soluble, and have an improved safety profile as compared to the AAV9 version. For reference, the wild type rh74 capsid used to generate these variants has a sequence of:

(SEQ ID NO: 4)
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL.

In a first variant, termed rh74Myo or rh74Myo-4E, the myotropic peptide replaces amino acids 588-590 of the rh74 capsid; i.e., in the recombinant capsid amino acid sequence position 587 of rh74 is followed by the myotropic peptide which in turn is followed by rh74 position 591. The full length capsid sequence is thus:

(SEQ ID NO: 1)
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQE
NRRGDFNNTAAPIVGAVNSQGALPGMVWQNRDVYLQGPIWAKIPHTDGN
FHPSPLMGGFGLKHPPPQILIKNTPVPADPPTTFNQAKLASFITQYSTG
QVSVEIEWELQKENSKRWNPEIQYTSNYYKSTNVDFAVNTEGTYSEPRP
IGTRYLTRNL.

The myotropic insert is underlined.

An exemplary nucleic acid encoding SEQ ID NO: 1 is:

(SEQ ID NO: 3)
atggctgccgatggttatcttccagattggctcgaggacaacctctctgagggcattcgcgagtggtgggacctgaaacctggag
ccccgaaacccaaagccaaccagcaaaagcaggacaacggccggggtctggtgcttcctggctacaagtacctcggaccctt
caacggactcgacaagggggagcccgtcaacgcggcggacgcagcggccctcgagcacgacaaggcctacgaccagcag
ctccaagcgggtgacaatccgtacctgcggtataatcacgccgacgccgagtttcaggagcgtctgcaagaagatacgtcttttg
ggggcaacctcgggcgcgcagtcttccaggccaaaaagcgggttctcgaacctctgggcctggttgaatcgccggttaagacg
gctcctggaaagaagagaccggtagagccatcaccccagcgctctccagactcctctacgggcatcggcaagaaaggccagc
agcccgcaaaaaagagactcaattttgggcagactggcgactcagagtcagtccccgaccctcaaccaatcggagaaccacca
gcaggcccctctggtctgggatctggtacaatggctgcaggcggtggcgctccaatggcagacaataacgaaggcgccgacg
gagtgggtagttcctcaggaaattggcattgcgattccacatggctgggcgacagagtcatcaccaccagcacccgcacctggg
ccctgcccacctacaacaaccacctctacaagcaaatctccaacgggacctcgggaggaagcaccaacgacaacacctacttc
ggctacagcaccccctgggggtattttgacttcaacagattccactgccacttttcaccacgtgactggcagcgactcatcaacaa
caactggggattccggcccaagaggctcaacttcaagctcttcaacatccaagtcaaggaggtcacgcagaatgaaggcacca
agaccatcgccaataaccttaccagcacgattcaggtctttacggactcggaataccagctcccgtacgtgctcggctcggcgca
ccagggctgcctgcctccgttcccggcggacgtcttcatgattcctcagtacgggtacctgactctgaacaatggcagtcaggct
gtgggccggtcgtccttctactgcctggagtactttccttctcaaatgctgagaacgggcaacaactttgaattcagctacaacttcg
aggacgtgcccttccacagcagctacgcgcacagccagagcctggaccggctgatgaaccctctcatcgaccagtacttgtact
acctgtcccggactcaaagcacgggcggtactgcaggaactcagcagttgctattttctcaggccgggcctaacaacatgtcgg
ctcaggccaagaactggctacccggtccctgctaccggcagcaacgcgtctccacgacactgtcgcagaacaacaacagcaa
ctttgcctggacgggtgccaccaagtatcatctgaatggcagagactctctggtgaatcctggcgttgccatggctacccacaag
gacgacgaagagcgattttttccatccagcggagtcttaatgtttgggaaacagggagctggaaaagacaacgtggactatagc
agcgtgatgctaaccagcgaggaagaaataaagaccaccaacccagtggccacagaacagtacggcgtggtggccgataac
ctgcaaGAAAACAGGAGAGGAGACTTCAACAACACTgccgctcctattgtaggggccgtcaatagt
caaggagccttacctggcatggtgtggcagaaccgggacgtgtacctgcagggtcccatctgggccaagattcctcatacggac
ggcaactttcatccctcgccgctgatgggaggctttggactgaagcatccgcctcctcagatcctgattaaaaacacacctgttcc
cgcggatcctccgaccaccttcaatcaggccaagctggcttctttcatcacgcagtacagtaccggccaggtcagcgtggagatc
gagtgggagctgcagaaggagaacagcaaacgctggaacccagagattcagtacacttccaactactacaaatctacaaatgtg
gactttgctgtcaatactgagggtacttattccgagcctcgccccattggcaccegttacctcaccegtaatctgtaa.

The insert sequence is capitalized. A person of skill in the art would recognize that, due to the redundancy of the genetic code, the above nucleotide sequence may vary significantly yet still encode the amino acid sequence of SEQ ID NO: 1.

A second variant places the myotropic insert into hyperviable region (HVR) IV (four) of the capsid, as opposed to the HVR-VIII where positions 587-591 are located. Both HVRs are in exposed loops of the AAV capsid protein and are amenable to mutation. In the HVR-IV variant, also called rh74 HVR4-4E, the myotropic insert replaces amino acids 452-461 such that a sequence of this region becomes 448 LSRTENRRGDFNNTQLL 464 (SEQ ID NO: 5). A full-length capsid sequence is thus:

(SEQ ID NO: 11)
MAADGYLPDWLEDNLSEGIREWWDLKPGAPKPKANQQKQDNGRGLVLPG
YKYLGPFNGLDKGEPVNAADAAALEHDKAYDQQLQAGDNPYLRYNHADA
EFQERLQEDTSFGGNLGRAVFQAKKRVLEPLGLVESPVKTAPGKKRPVE
PSPQRSPDSSTGIGKKGQQPAKKRLNFGQTGDSESVPDPQPIGEPPAGP
SGLGSGTMAAGGGAPMADNNEGADGVGSSSGNWHCDSTWLGDRVITTST
RTWALPTYNNHLYKQISNGTSGGSTNDNTYFGYSTPWGYFDFNRFHCHF
SPRDWQRLINNNWGFRPKRLNFKLFNIQVKEVTQNEGTKTIANNLTSTI
QVFTDSEYQLPYVLGSAHQGCLPPFPADVFMIPQYGYLTLNNGSQAVGR
SSFYCLEYFPSQMLRTGNNFEFSYNFEDVPFHSSYAHSQSLDRLMNPLI
DQYLYYLSRTENRRGDFNNTQLLFSQAGPNNMSAQAKNWLPGPCYRQQR
VSTTLSQNNNSNFAWTGATKYHLNGRDSLVNPGVAMATHKDDEERFFPS
SGVLMFGKQGAGKDNVDYSSVMLTSEEEIKTTNPVATEQYGVVADNLQQ
QNAAPIVGAVNSQGALPGMVWQNRDVYLQGPIWAKIPHTDGNFHPSPLM
GGFGLKHPPPQILIKNTPVPADPPTTFNQAKLASFITQYSTGQVSVEIE
WELQKENSKRWNPEIQYTSNYYKSTNVDFAVNTEGTYSEPRPIGTRYLT
RNL.

The myotropic insert is underlined.

A third capsid variant has HVR-IV of rh74Myo replaced with the corresponding sequence of AAV9, as the HVRs are oriented close to one another in the capsid three-dimensional structure. The HVR-IV of this variant (rh74Myo.HVR4-AAV9) thus becomes

(SEQ ID NO: 6)
448 LSRTINGSGONQQTLL 460

The AAV9 amino acids are underlined. Note that the HVR-IV of AAV9 is one amino acid shorter than the corresponding region of rh74.

A fourth variant has the myotropic peptide inserted in both HVR-IV and HVR-VIII, as described above for the first two variants. This variant is termed rh74Myo.HVR4-4E.

The efficacy and safety of the four myotropic rh74 capsid variants for MHCK7.NHP-μDys construct delivery to DMD^{mdx} mice were evaluated. Four to six week old mice were treated with the capsid variants intravenously via tail vein injections. Four and eight weeks after treatment, the mice were analyzed for vector biodistribution: expression of microdystrophin by RNA, western blot and immunofluorescence; TA (tibialis anterior) force; histopathology; and anti-capsid antibodies. The experimental groups are given in Table 1.

TABLE 1
Evaluation of myotropic rh74 variants

| | Capsid | HVR- | HVR- | Dose |
|---|---|---|---|---|
| Capsid | Backbone | IV | VIII | (vg/kg) |
| Rh74Myo-4E | rh74 | rh74 | 4E | 2.25 × 10¹³ |
| Rh74Myo4E.HVR4-4E | rh74 | 4E | 4E | 2.19 × 10¹³ |
| Rh74 hd | rh74 | rh74 | rh74 | 1.33 × 10¹⁴ |
| Rh74.HVR4-4E | rh74 | 4E | rh74 | 2.19 × 10¹³ |
| MyoAAV-4E hd | AAV9 | AAV9 | 4E | 4 × 10¹³ |
| Rh74Myo.HVR-AAV9 | rh74 | AAV9 | 4E | 2.19 × 10¹³ |
| MyoAAV-4E ld | AAV9 | AAV9 | 4E | 2 × 10¹³ |
| Rh74 ld | rh74 | rh74 | rh74 | 2 × 10¹³ |

hd = high dose; ld = low dose.

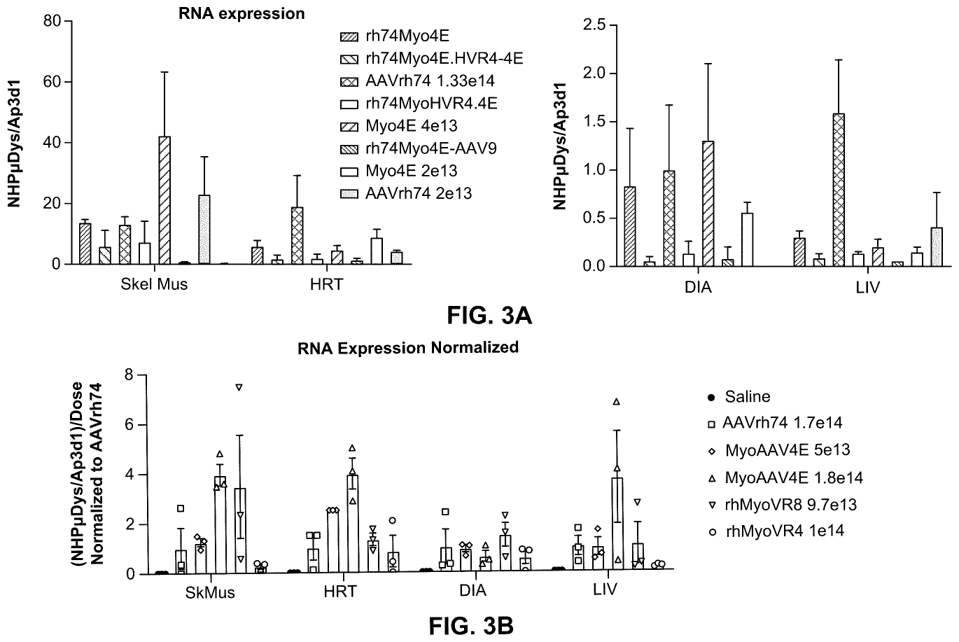

Representative data is presented in FIGS. 3A-3B. Shown is microdystrophin RNA expression at 4 weeks in skeletal muscle, heart, diaphragm, and liver (FIG. 3A). Both the rh74Myo and the rh74.HVR4-4E variants were able to achieve at least comparable microdystrophin expression as compared to higher doses of wild type rh74. A second experiment evaluated just these two variants (data normalized to the rh74 dose to facilitate more direct comparison (FIG. 3B)). The HVR-VIII version, rh74Myo, displayed the greater improvement in muscle targeting as compared with the wild type capsid.

### Example 3: Comparison of rh74 Myo to a MyoAAV on the AAV9 Backbone

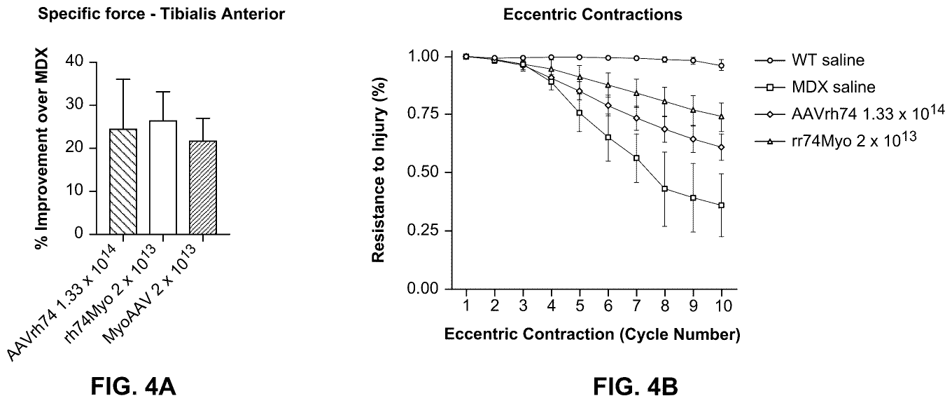

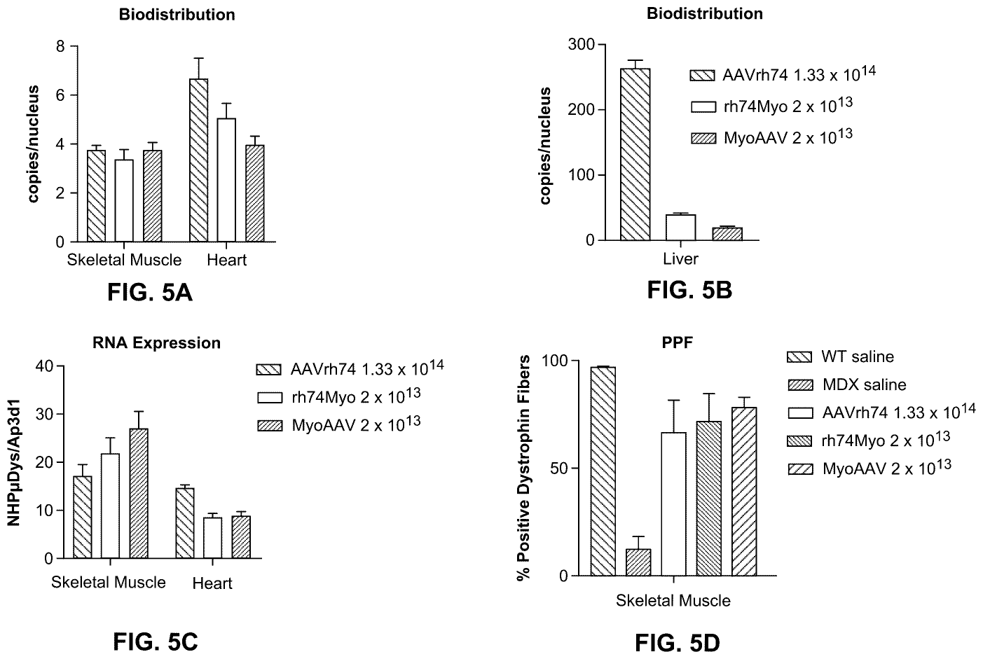

We evaluated the efficacy and safety of two myotropic capsid variants, rh74Myo and MyoAAV, in DMD^{mdx} mice (FIGS. 4A-4B) and nonhuman primates (NHPs; FIGS. 5A-5D).

A myotropic peptide sequence was inserted into hypervariable region VIII of AAVrh74 and AAV9 for MHCK7.NHP-μDys construct delivery (rh74Myo and MyoAAV, respectively). Biodistribution and efficacy were evaluated in DMDmdx mice administered intravenous (IV) AAVrh74 (1.33×10¹⁴ vg/kg), rh74Myo (2×10¹³ vg/kg), or MyoAAV (2×10¹³ vg/kg). Bio-distribution and safety were evaluated in NHPs (cynomolgus macaques) administered IV AAVrh74 (2×10¹⁴ vg/kg), rh74Myo (1×10¹⁴ vg/kg), or MyoAAV (5×10¹³, 7×10¹³, 2×10¹⁴ vg/kg).

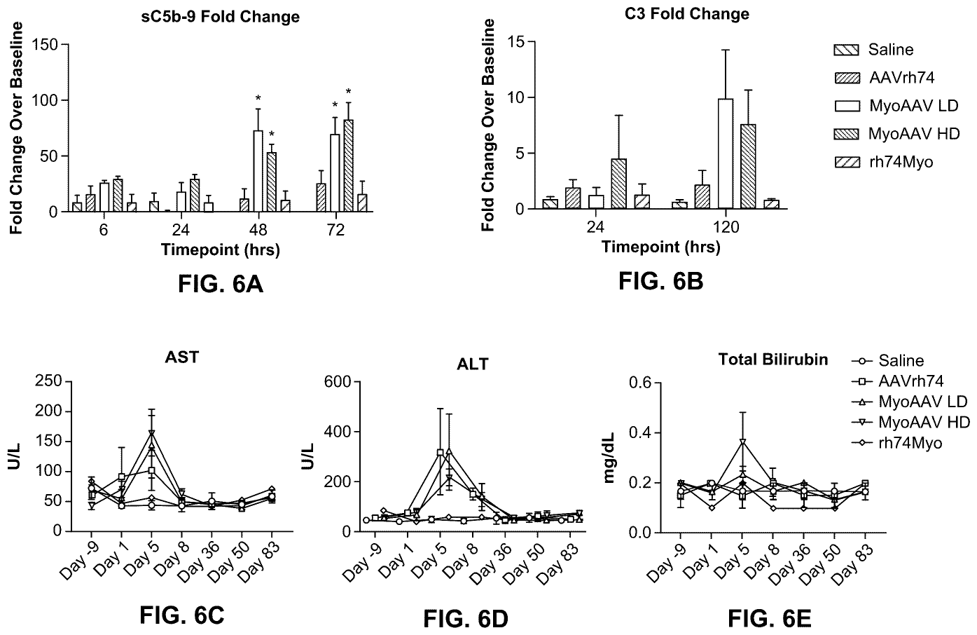

At >6-fold lower dose than AAVrh74, both rh74Myo and AAV9-based-MyoAAV restored tibialis anterior muscle function as measured by specific force (FIG. 4A) and injury resistance (FIG. 4B), and both produced skeletal muscle NHP-μDys expression comparable to higher-dose AAVrh74 in DMD^{mdx} mice, with a corresponding >6-fold decrease in liver biodistribution. In NHPs, skeletal muscle transgene delivery/μDys expression were enhanced with rh74Myo and MyoAAV compared with AAVrh74, as measured by biodistribution (FIG. 5A), RNA expression (FIG. 5C) and percent positive muscle fibers (FIG. 5D). As with the mice, both displayed decreased liver biodistribution in NHPs (FIG. 5B) No test article-related pathology or immune activation were noted with rh74Myo. Complement pathways (FIGS. 6A-6B) and serum liver enzymes (FIGS. 6C-6E) were elevated following MyoAAV; AAVrh74 and rh74Myo were not associated with elevated complement. A complement activation event with significantly increased serum liver enzymes and decreased platelet counts was detected with AAV9-based-MyoAAV (7×10¹³ vg/kg).

Insert of a myotropic peptide (SEQ ID NO: 2) into an AAV9 capsid enhances muscle targeting, but the variant capsid is less stable, tends to aggregate, and produces poorly. In contrast, inserting the same peptide into either rh74 HVR-IV or HVR-VIII (both not both) does not destabilize the capsid variants nor does the modification cause complement activation or elevated serum liver enzymes. The myotropic capsid variant rh74Myo HVR-VIII enhanced skeletal muscle transduction without increasing hepatic targeting and has a favorable safety profile similar to AAVrh74, supporting further clinical development for skeletal muscle disorders.

### Example 4. Formulation Buffers

Buffers are important to the production and use of rAAV vectors as the buffer should stabilize and preserve the integrity of rAAV and prevent loss due to aggregation, absorption to surfaces and degradation.

The rAAV disclosed herein may be present in compositions of approximately 20 mM Tris [tris(hydroxymethyl)aminomethane] with about 1 mM MgCl₂ [magnesium chloride] and about 180-200 mM NaCl [sodium chloride] at about pH 8. The composition may further contain poloxamer or polysorbate 80 as a surfactant, emulsifier, solubilizer, and/or dispersing agent. Poloxamer was tested at about 0.001% volume/volume to about 0.2%. One such poloxamer is termed P188, i.e., a poloxamer having a molecular weight of 188 kDa, although other poloxamers have been used in pharmaceutical compositions. Patel, Int'l J. Pharmtech Res. 1 (2): 299 (2009). Polysorbate 80 was tested at 0.0001% to 0.005%. Although all ranges of surfactant tested improved recovery of the rAAV as compared to pharmaceutical compositions lacking surfactant, the higher values were generally more efficacious.

The composition may further contain sucrose. One property of sucrose is to minimize freeze/thaw stress which may lead to capsid breakage or degradation. Sucrose was tested at 1%, 2%, 3%, 4%, 5%, 6%, 7%, 8%, 9% and 10% weight/volume. Other cryoprotectants may be trehalose, lactose, sorbitol, glycerol, (poly)ethylene glycol, or mannitol.

One exemplary formulation would be 20 mM Tris pH 8, 1 mM MgCl₂, 180-200 mM NaCl, 0.1% P188 and 1-3% sucrose. Another exemplary formulation would be 20 mM Tris pH 8, 1 mM MgCl₂, 180-200 mM NaCl, and 0.01% P188. The formulation for rh74Myo is 20 mM Tris pH 8, 1 mM MgCl₂, 180 mM NaCl, 0.1% P188 and 2% sucrose.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A modified AAV particle comprising (i) a capsid protein comprising an AAVrh74 capsid and a peptide comprising the amino acid sequence set forth in SEQ ID NO: 2; and (ii) a transfer cassette comprising a 5′ ITR, a kazak sequence, a chimeric intron, an MHCK7 promoter, a microdystrophin gene, a polyA sequence, and a 3′ ITR,
wherein the microdystrophin gene comprises the nucleotide sequence as set forth in SEQ ID NO: 8.

2. The modified AAV particle of claim 1, wherein the peptide is located between amino acids corresponding to amino acids 587 and 591 of SEQ ID NO: 4 and amino acids corresponding to amino acids 588, 589, and 590 of SEQ ID NO: 4 are absent.

3. The modified AAV particle of claim 1 or 2, wherein the capsid protein comprises the amino acid sequence as set forth in SEQ ID NO: 1.

4. The modified AAV particle of claim 3, wherein the capsid protein is encoded by the nucleotide sequence as set forth in SEQ ID NO: 3.

5. (canceled)

6. The modified AAV particle of claim 1, wherein the transfer cassette comprises the nucleotide sequence as set forth in SEQ ID NO: 17.

7. A pharmaceutical composition comprising the modified AAV particle of claim 1, and a pharmaceutically acceptable carrier.

8.-9. (canceled)

10. A method of treating a muscular disease comprising administering to a subject in need thereof a therapeutically effective amount of the pharmaceutical composition of claim 7.

11. The method of claim 10, wherein the muscular disease is Duchenne muscular dystrophy, Becker's muscular dystrophy, Myotonic muscular dystrophy, Facioscapulohumeral muscular dystrophy, a limb-girdle muscle dystrophy, or a cardiomyopathy.

12. (canceled)

13. A pharmaceutical composition comprising (i) about 20 mM Tris, (ii) about 1 mM MgCl₂, (iii) about 180-about 200 mM NaCl, (iv) about 0.01%-about 0.2% poloxamer, (v) an AAV particle, and (vi) optionally about 1%-about 3% sucrose.

14. (canceled)

15. The pharmaceutical composition of claim 13, wherein (i) the NaCl is at a concentration of about 180 mM, about 190 mM, or about 200 mM, (ii) the poloxamer is at a concentration of about 0.01%, about 0.02%, about 0.03%, about 0.04%, about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, or about 0.2%, and/or (iii) the sucrose is at a concentration of about 1%, about 1.5%, about 2%, about 2.5%, or about 3%.

16.-18. (canceled)

19. The pharmaceutical composition of claim 13, comprising (i) about 20 mM Tris, (ii) about 1 mM MgCl₂, (iii) about 180 NaCl, (iv) about 0.01% poloxamer, and (v) an AAV particle.

20. The pharmaceutical composition of claim 13, comprising (i) about 20 mM Tris, (ii) about 1 mM MgCl₂, (iii) about 180 NaCl, (iv) about 0.01% poloxamer, (v) about 2% sucrose, and (vi) an AAV particle.

21.-26. (canceled)

27. The pharmaceutical composition of claim 13, wherein the AAV particle comprises a capsid protein and a transfer cassette, wherein the capsid protein comprises a peptide targeting muscle comprising the amino acid sequence as set forth in SEQ ID NO: 2.

28. The pharmaceutical composition of claim 27, wherein the peptide is located in Hyper Variable Region VIII of a Rh74 capsid protein or Hyper Variable Region IV of a Rh74 capsid protein.

29. The pharmaceutical composition of claim 27, wherein the peptide is located (i) between amino acids corresponding to amino acids 587 and 591 of a wild type AAVRh74 capsid protein, and amino acids corresponding to amino acids 588, 589, and 590 of a wild type AAVRh74 are absent or (ii) between amino acid residues corresponding to amino acids 451 and 462 of a wild type AAVRh74 capsid protein, and wherein amino acid residues corresponding to amino acids 452-461 of a wild type AAVRh74 capsid protein are absent.

30.-32. (canceled)

33. A method of treating a disease or condition in a subject in need thereof comprising administering to the subject the pharmaceutical composition of claim 13.

34. The method of claim 33, wherein the disease or condition comprises a muscular disease.

35. The method of claim 34, wherein the muscular disease is Duchenne muscular dystrophy, Becker's muscular dystrophy, Myotonic muscular dystrophy, Facioscapulohumeral muscular dystrophy, a limb-girdle muscle dystrophy, or a cardiomyopathy.

36. The method of claim 33, wherein the subject is a human.

37. A modified AAV particle comprising (i) a capsid protein comprising the amino acid sequence as set forth in SEQ ID NO: 1 or SEQ ID NO: 11 and (ii) a transfer cassette comprising the nucleotide sequence as set forth in SEQ ID NO: 17.

38.-40. (canceled)
